(19) United States
(12) Patent Application Publication
(10) Pub. No.: US 2025/0332282 A1
(43) Pub. Date: Oct. 30, 2025

(54) FACET-BASED NUCLEIC ACID PLATONIC, KEPLER-POINSOT POLYHEDRA AND FOUR-DIMENSIONAL TESSERACT FOR THERAPEUTIC, DIAGNOSTIC AND ANALYTICAL APPLICATIONS

(71) Applicant: The University of Hong Kong, Hong Kong (HK)

(72) Inventors: Julian Alexander TANNER, Hong Kong (HK); Simon Chi-Chin SHIU, Hong Kong (HK); Nicholas Xuanlai FANG, Hong Kong (HK)

(21) Appl. No.: 19/189,356

(22) Filed: Apr. 25, 2025

Related U.S. Application Data
(60) Provisional application No. 63/638,955, filed on Apr. 26, 2024.

(51) Int. Cl. A61K 47/69; A61K 45/06; G01N 33/543

(52) U.S. Cl. CPC A61K 47/6925; A61K 45/06; G01N 33/54346

(57) ABSTRACT

Facet-based DNA polyhedral nanostructures with single strands of DNA for each polyhedron face. The DNA-based polyhedral nanostructures may be configured for targeted drug delivery and cell imaging for therapeutic, diagnostic and analytical diagnostic applications. The DNA polyhedral nanostructures adopt platonic icosahedral and dodecahedral configurations resembling the natural shapes of viral nucleocapsid proteins. The DNA polyhedral nanostructures address multiple technical challenges in the biomedical and biotechnological fields, including reducing the cost and complexity associated with conventional DNA origami systems, enhancing the sensitivity of diagnostic platforms, improving tracer delivery and signal clarity in PET and MRI, providing structural stability for nanostructure and payload integrity, and improving the resolution of Cryo-EM imaging of small molecules.

### CROSS-REFERENCE TO RELEVANT APPLICATIONS

The present application claims priority from a U.S. Provisional Patent Application No. 63/638,955 filed Apr. 26, 2024, and the disclosure of which are incorporated by reference in their entirety.

### REFERENCE TO SEQUENCE DISCLOSURE

The sequence listing file under the file name “P3192US01_seq listing.xml” submitted in ST.26 XML file format with a file size of 63 KB created on Apr. 25, 2025 and filed on Apr. 25, 2025 is incorporated herein by reference.

### FIELD OF THE INVENTION

The present invention relates to DNA geometrical rearrangement. In particular, a method of facet-based DNA polyhedra fabrication is disclosed, allowing the construction of various polyhedral forms including icosahedron, dodecahedron, Kepler-Poinsot polyhedron and tesseract.

### BACKGROUND

In the atomic world, elements co-exist in the form of compounds forming various molecules with different shapes. The most versatile type of molecules is the organic hydrocarbon that can even form a buckminsterfullerene C₆₀.

As the field of chemistry and molecular engineering evolves alongside technological advancements, molecules with different shapes have been created in chemistry except tesseract (also known as hypercube), a hypothetical structure which is a three-dimensional visualized projections of a four-dimensional analog of a cube as a “cube inside a cube connected at the corners”.

Similar, there have been numerous attempts at morphing DNAs into polyhedra, also referred to as “DNA origami”. DNA origami is realizable due to the intrinsic base-pairing rules of DNA, i.e. adenine-thymine and guanine-cytosine nucleotide pairing, which allows atomic-level precision in building two-dimensional or even three-dimensional shapes by programming specific sequences that pair up, fold and self-assemble into predictable structures.

DNA origami is a powerful bio-nanotechnology tool. With flexible and customizable structuring, coupled with the intrinsic biocompatibility of DNA, DNA origami has immense potentials across a plethora of fields, ranging from high-precision drug delivery, cell scaffolding and more.

Conventional DNA nanostructure assembly techniques rely heavily on top-down approaches, in which a preselected target geometry is computationally modeled and then rendered as a folded configuration of a long, single-stranded DNA scaffold. The scaffold is folded into the desired shape using hundreds of short complementary “staple” strands that hybridize with specific segments of the scaffold. This technique, known as DNA origami, enables the construction of two- and three-dimensional nanostructures with high geometric complexity and precision.

However, top-down DNA origami approaches present several limitations. The design of intricate shapes requires significant computational resources to route the scaffold through a desired geometric path without sequence collisions or overlaps. Each new structure generally requires a redesign of the entire staple library, leading to high synthesis costs and reduced scalability. Additionally, the dependency on a fixed-length scaffold (often derived from M13 bacteriophage DNA) imposes constraints on shape modularity, size, and sequence customization. The necessity of hundreds of staple strands also complicates purification and reduces robustness in low-concentration or multiplexed systems.

Furthermore, in traditional scaffold-based methods, the folding pathway of the structure is not readily programmable via strand-to-strand interactions alone. Instead, it requires global scaffold routing and artificial junction design, which can limit dynamic reconfiguration, parallel assembly, or integration with other bottom-up nanoscale systems.

One example of this scaffold-centric approach is described in U.S. Pat. No. 11,410,746 B2, which discloses nucleic acid assemblies constructed by routing a scaffold strand through a geometrically modeled node-edge framework. In that approach, a spanning-tree-based algorithm is used to assign scaffold paths and design crossover points, with staple strands facilitating local folding. While the patent notes that staples may be omitted in some configurations, the overall design still fundamentally depends on a long scaffold strand that is computationally routed through the structure. As such, the assembly remains scaffold-based and does not eliminate the complexity or cost associated with scaffold and staple synthesis.

Other prior work has attempted scaffold-free or partially modular designs by assigning oligonucleotides to geometric features of the structure, such as the He et al. study (Chem. Commun., 2013, 49, 2906), which describes the construction of a DNA triangular prism using either a single ssDNA strand that winds around the edges, or multiple short strands that partially encircle each face. In that design, each oligonucleotide participates in forming double-helical edges and contributes to more than one face or structural edge, and hybridization occurs along extended paths.

However, the difficulty of DNA origami production increases drastically with increasing structural complexity. To start with, as polyhedral complexity increases, the number of edges, vertices and junctions also grows exponentially. This in turn poses great computational intensity as not only must the strand routing avoid overlaps, knots or breaks, but any minute error down to a mispaired base may cause the entire structure to fail.

It should also be noted that, given the reliance of the entire DNA origami on a long scaffold strand of thousands of bases to weave through the entire structure, routing the scaffold through a complex geometry, more so for multi-faced polyhedra, is difficult as any misplaced crossover leads to local strain, incomplete closure or incorrect folding, and eventually collapse of the three-dimensional shape.

As such, there is a need in the field for a more reliable and simpler synthesis method to overcome the technical hurdles of DNA polyhedra formation. The present invention addresses this need.

### SUMMARY OF THE INVENTION

The present invention addresses the limitations of scaffold-based and staple-driven DNA nanostructure assembly by providing a modular, bottom-up self-assembly method for constructing closed polyhedral structures using a plurality of single-stranded DNA (ssDNA) oligonucleotides, each of which corresponds to a discrete face of the polyhedron. Each ssDNA oligonucleotide includes one or more edge-specific hybridization domains, designed to selectively hybridize with complementary domains on ssDNA strands defining adjacent faces.

Unlike scaffold-based origami techniques, the disclosed system is scaffold-free and staple-free. The ssDNA strands self-assemble by localized hybridization at edge regions, forming a structurally closed and topologically well-defined nanostructure without the need for a pre-routed backbone or externally induced folding. Each face-specific oligonucleotide is uniquely configured such that it hybridizes only with the correct adjacent strands, supporting both programmability and high-fidelity construction. No strand spans more than one face, and no scaffold or staple strands are required. This architecture enables true bottom-up assembly of closed DNA polyhedra using a minimal, programmable set of oligonucleotides.

In certain embodiments, surface acoustic waves (SAWs) may be applied to promote spatial clustering and enhance hybridization efficiency. The disclosed architecture allows for the efficient construction of nanoscale cubes, tesseracts, and other polyhedral forms with minimal strand count and design overhead, and is suitable for applications in nanoelectronics, sensing, and programmable self-assembly.

In one aspect, the method involves: (i) mixing equimolar amounts of single-stranded DNAs with water to obtain 100 μM working solutions of individual components of the polyhedral structure, wherein each component is one face of the polyhedron; (ii) mixing each of the 100 μM working solutions of individual components of the polyhedral structure in phosphate buffer saline to obtain pre-assembly solutions, wherein the phosphate buffer saline comprises 140 mM NaCl, 3 mM KCl and 10 mM phosphate buffer; (iii) mixing all the pre-assembly solutions to obtain an assembly mixture; and (iv) annealing the assembly mixture in a thermal cycler by incubating at 95° C. for 2 to 5 minutes then slowly annealed from 95° C. to 20° C. at the rate of 4 to 5° C./hour to obtain the folded structure of the DNA polyhedron.

In an embodiment of the present invention, the shape of the DNA polyhedron is selected from icosahedron, dodecahedron, tesseract or any K epler-Poinsot polyhedron.

In another embodiment, the DNA polyhedron formed by the method of the present invention has a melting temperature of at least 80° C.

In another embodiment, the DNA polyhedron further comprises inner paranemic crossover (Px) motifs.

### BRIEF DESCRIPTION OF THE DRAWINGS

FIGS. 1A to 1C demonstrates the design of DNA tesseract of the present invention. FIG. 1A shows the components of the DNA tesseract. FIG. 1B is a schematic of the DNA tesseract from multiple perspectives. FIG. 1C shows electron density maps of the DNA tesseract determined by Cryo-EM single particle analysis.

FIGS. 2A to 2H demonstrate the assembly of DNA Tesseract and stabilization of small cube. FIG. 2A shows the formation of the four components of DNA tesseract. FIG. 2B displays the partial assemblies (upper) and signal of fluorescence resonance energy transfer from the small cube of DNA tesseract (lower). FIG. 2C shows fluorescent plate assay for quantitative measurement of FRET signal from small cube in corresponding combinations. FIG. 2D shows atomic force microscopy of DNA tesseract. FIG. 2E shows representative micrograph of Cryo-EM of DNA tesseract and 2D classifications (particles that resembled the shape of tesseract are picked with topaz trained model in CryoSPARC and the 2D classifications revealed the three major angles as in FIG. 1). FIG. 2F shows the cryo-EM 3D resolution map of the reconstruction of DNA tesseract with C1 symmetry. FIG. 2G shows the 3D resolution map of the reconstruction of DNA tesseract with octahedral symmetry. FIG. 2H shows the size characterization of the DNA tesseract.

FIG. 3 illustrates the thermal stability of DNA tesseract and the partial assemblies using qPCR and circular dichroism.

FIGS. 4A to 4C illustrates several designs of DNA polyhedra of the present invention. FIG. 4A shows the strands layout of the DNA icosahedron; and FIG. 4B and FIG. 4C respectively shows the layouts of great icosahedron, great stellated dodecahedron (FIG. 4B), great dodecahedron and small stellated dodecahedron (FIG. 4C), the four of which are collectively known as K epler-Poinsot polyhedra.

FIG. 5 illustrates the DNA icosahedron with inner Px motif framework with golden ratio for enhancing rigidity.

FIG. 6 illustrates the DNA tesseract.

FIG. 7 shows the gel electrophoresis showing the formation of icosahedron, dodecahedron and icosahedron with Px motif at different concentrations.

FIG. 8 displays the gel electrophoresis showing the formation of Kepler-Poinsot polyhedra at different concentrations.

FIGS. 9A and 9B shows microscopic observations of icosahedron, both unfolded and folded. FIG. 9A shows the transmission electron microscopy image; and FIG. 9B shows the atomic force microscopy of the unfolded and folded icosahedra, respectively.

FIG. 10 shows microscopic observation of the dodecahedron with transmission electron microscopy and atomic force microscopy.

FIG. 11 shows microscopic observation of Kepler-Poinsot polyhedra with transmission electron microscopy and atomic force microscopy.

FIG. 12 illustrates the Cryo-EM reconstruction of DNA icosahedron.

FIG. 13 shows the gel electrophoresis of the big cube, small cube and trapezoidal prisms A and B respectively, demonstrating the assembly of each part of the tesseract.

FIG. 14 shows the EM SA analysis of all 15 possible sub-structure combinations of the DNA tesseract on gel electrophoresis.

FIG. 15 shows serial dilution of DNA tesseract at different concentrations.

FIG. 16 shows atomic force microscopy observation of DNA Tesseract.

FIG. 17 illustrates a mechanism of microscale assembly of the DNA tesseract into a long wire.

FIGS. 18A and 18B demonstrate FRET observation of small cube assembly within the DNA tesseract observed by agarose gel electrophoresis. FIG. 18A shows FRET signal observed on the gel for investigating the small cube structural integrity. FIG. 18B shows the diagonal situating of Cy3 and Cy5 FRET pairs on the small cube to allow observation of small cube assembly within the tesseract structure. Blue sphere: Cy3; Green sphere: Cy5.

FIG. 19 shows the correlation function of the DLS measurement of DNA tesseract in FIG. 2H.

FIGS. 20A to 20C show the force-distance spectra of the DNA tesseract and its components respectively. FIG. 20A shows the force-distance spectra of big cube and tesseract. FIG. 20B shows the individual force-distance spectra of 10 random particles that contributed to the mean spectrum of the big cube in FIG. 20A. Spectra are obtained by approaching the AFM tip towards the particle followed by retraction. Significant adhesion was consistently observed across 10 measurements, indicating the weakness in resisting expansion. FIG. 20C show the individual force-distance spectra of 10 random particles that contributed to the mean spectrum of the tesseract in FIG. 20A. Spectra are obtained by approaching the AFM tip towards the particle followed by retraction. As compared to FIG. FIG. 20B, no adhesion was observed, indicating the comparative strength in resisting expansion.

FIGS. 21A and 21B show the nuclease resistance of the DNA tesseract observed by FRET signal on agarose gel electrophoresis. FIG. 21A shows the titration of 15 nM DNA tesseract with increasing concentrations of DNase I with a wide range of concentrations (left) and a narrow range of concentrations (right). FIG. 21B shows incubation of 15 nM DNA tesseract and a duplex (same distance between the FR ET pair) in 95% FBS. Blue sphere: Cy3; Green sphere: Cy5.

FIGS. 22A to 22I displays the assembly of DNA tesseract into micro-scale wire for electrical conduction. FIG. 22A illustrates the principle of using surface acoustic wave (SAW) to locally concentrate DNA tesseract for clustering. FIG. 22B shows the design of tesseract with complementary anchors. Duplexes are added on top of the original design to connect two tesseracts in diagonal manner. FIG. 22C displays a zoomed-in image from brightfield microscopy observing the presence of DNA tesseract clusters in the glass capillary after application of SAW (342 MHz, 2 V, burst period 30 ms, 1×10⁶ cycles, 30 minutes). Scale bar: 10 μm. FIG. 22D is a compilation of diameters of 1,239 particles measured with an automated image process implemented in MATLAB. Average diameter is 1.93 μm and median is 1.55 μm. Error bar indicates standard error. FIG. 22E shows a representative image of tesseract wire on IDE. Scale bar: 20 μm. FIG. 22F shows an SEM image of the same wire in FIG. 22E. Scale bar: 20 μm. FIG. 22G is a confocal image of a tesseract wire stained with Cy3 anchor. Scale bar: 100 μm. FIG. 22H shows the measurement of conductivity on IDE. Three different structures before and after the application of SAW are tested. FIG. 22I shows the reciprocal of the data in h reflecting the resistance.

FIGS. 23A and 23B are representative images from brightfield microscope observing the formation of aggregates upon the application of SAW. FIG. 23A shows the formation of tesseract aggregate; and FIG. 23B shows the observation of aligned particles in the capillary after the application of SAW.

FIG. 24 shows confocal microscopy images of IDE. IDEs with observable wire from FIG. 26 were stained with 10 μl of 1 μM Cy3 anchor for imaging. Cy3 signal was observed at where a wire could be identified under brightfield microscope.

FIG. 25 shows energy dispersive X-ray spectroscopy in SEM. Elements on the IDE were mapped to identify the presence of DNA. Blank is mapped to observe the presence of background element.

FIG. 26 displays representative brightfield microscopy micrographs of IDE. The electrodes were deposited with PBS, tetrahedron, BcA or tesseract. Wire was only observed for samples with SAW applied. Scale bar: 100 μm.

FIG. 27 displays the DNA melting of tesseract and components by circular dichroism, as an extension to FIG. 3. CD spectra are scanned from 220 nm to 320 nm with an increase in temperature from 20° C. to 95° C. The signature of DNA duplex is observed with a negative peak at 247 nm and a positive peak at 277 nm. As temperature increases, the CD signal reduces.

FIG. 28 displays the qPCR melting of DNA tesseract at different concentrations as an extension to FIG. 3. Distinguishable peak value was observable starting from 50 nM onwards

FIGS. 29A and 29B show 3D fourier shell correlation determining resolution of reconstruction of DNA tesseract. FIG. 29A shows the 3D fourier shell correlation for DNA tesseract reconstruction with C1 symmetry. FIG. 29B shows the 3D fourier shell correlation for DNA tesseract reconstruction with octahedral symmetry. The resolution determined for C1 and octahedral symmetries were 21.24 Å (FIG. 29A) and 14.16 Å (FIG. 29B) respectively.

### DETAILED DESCRIPTION

As used herein, the term “tesseract” refers to the three-dimensional projection of a four-dimensional hypercube, visualized as a cube enclosed within another cube, interconnected at corresponding vertices by trapezoidal prism structures.

As used herein, the term “Kepler-Poinsot polyhedron” refers to any of the four regular star polyhedra, including great dodecahedron, small stellated dodecahedron, great icosahedron and great stellated dodecahedron, each formed by stellating the regular convex dodecahedron and icosahedron.

As used herein, the term “paranemic crossover motif” and its abbreviated form “Px motif” refers to a structural arrangement of two parallel double-stranded DNA helices connected at periodic intervals via non-covalent crossover interactions without any base-pairing interruption. In general, these motifs contribute to structural rigidity by maintaining specific dihedral angles, consistent edge lengths and twist per edge within DNA polyhedra.

As described above, while there is immense application potential for DNA polyhedral nanostructures across different fields including but not limited to biomedical application, and diagnostics and therapeutics, the formation of DNA polyhedra remains highly complex and requires highly precise assembly to avoid structural failure, thereby limiting their practical application in biomedical and diagnostic contexts.

The present invention provides a method for assembling artificial DNA polyhedra using single-stranded DNA, each representing one face of the desired polyhedral structure. Assembly occurs through Watson-Crick base pairing, with controllable polyhedron size through adjusting strand length.

In one aspect In one embodiment, the polyhedral nanostructure is assembled from a plurality of single-stranded DNA oligonucleotides, each representing one face of the polyhedron. For example, in the case of a cube, six unique ssDNA strands may be used, each defining a square face.

Edge hybridization domains are located at or near 5′ and 3′ termini or distributed along the edges. These edge domains are designed to be complementary only to specific domains on ssDNA strands defining adjacent faces. For example, the edge domain of the strand corresponding to the top face may hybridize only with the top-facing domains of the front, back, left, and right faces.

Hybridization occurs when the strands are incubated under conditions favorable for selective Watson-Crick base pairing, such as in a controlled hybridization buffer with defined salt concentration and temperature. Once edge domains hybridize, the polyhedral structure closes via face-to-face connection at edges, with no scaffold or external staples involved in folding or stabilization.

In some embodiments, the total length of each ssDNA oligonucleotide ranges from approximately 20 to 200 nucleotides, substantially smaller than large, single strand DNA that creates an entire polyhedron or that wraps around multiple polyhedron edges. However, it is understood that the approach of the present invention (one DNA strand per face) is scalable to longer DNA lengths when fabricating larger polyhedral nanostructures.

The resulting structure is a staple-free, scaffold-free polyhedron with addressable faces and edges, capable of integration into higher-order lattices, nanoscale circuitry, or programmable nanosystems.

The DNA polyhedral nanostructures in the present invention are configured for use in targeted drug delivery and cell imaging for therapeutic, diagnostic and analytical diagnostic applications. These structures adopt geometric configurations such as simple platonic icosahedra and dodecahedra, resembling the natural shapes of viral nucleocapsid proteins, while allowing tunability in structural design.

A molecular engineering method of constructing an artificial DNA polyhedron, comprising: (i) providing a plurality of single-stranded DNA (ssDNA) oligonucleotides, wherein each ssDNA is engineered to correspond to one face of the polyhedron; (ii) mixing equimolar amounts of each ssDNA to form pre-assembly mixtures; (iii) mixing all pre-assembly mixtures to obtain an assembly mixture; and (iv) annealing the assembly mixture by thermal cycling, comprising heating to denaturation, and controlled cooling for Watson-Crick base pairing between complementary regions of the ssDNAs to assemble a folded structure of the DNA polyhedron.

Using this method, a DNA tesseract is assembled, comprising a smaller cube enclosed within a larger cube and connected via trapezoidal prisms.

A wireframe design is employed, wherein each face of the structure is composed of a single strand on each face of each component, and the only strand breaks are positioned at the vertices. The formation of the internal occurs only upon integration with surrounding structural components, as confirmed by Cryo-EM imaging.

The DNA tesseract demonstrates a melting temperature of up to 84.5° C. and the small cube is spatially fixed within the tesseract. The hypercube structure stabilized the four single-stranded DNA which are otherwise unstable to form the internal cube. The creation of DNA tesseract offers potential for applications across areas including chemistry, material science and medicine.

The DNA tesseract is further characterized by its scalability, as each tesseract require as few as 16 oligonucleotides to form, and the applicability of surface acoustic waves for clustering of the tesseracts at low concentrations enable the present invention as a potential substitute over existing DNA origami technology for complex DNA-based electronic circuitry and other applications.

The DNA polyhedra of the present invention address key limitations in nanostructure-based drug delivery, diagnostics, molecular imaging, structural stability and resolution in cryo-electron microscopy.

The method of the present invention is applicable to forming additional polyhedral configurations, including but not limited to icosahedrons, dodecahedrons, Kepler-Poinsot polyhedra, and other Platonic polyhedra.

### EXAMPLES

### Example 1: Method of DNA Tesseract Formation and Analysis

### 1.1 Design and Assembly of DNA Tesseract

In general, method of facet-based DNA polyhedron formation of the present invention involves mixing equimolar amounts of single-stranded DNAs in water to obtain 100 μM working solutions; mixing all the 100 μM working solutions with phosphate buffer saline comprising 140 mM NaCl, 3 mM KCl and 10 mM phosphate buffer to obtain pre-assembly solutions, mixing all the pre-assembly solutions to obtain an assembly mixture and annealing the assembly mixture in a thermal cycler by incubating at 95° C. for 2 to 5 minutes then through a controlled cooling phase at a rate of 4 to 5° C./hour to a final temperature 20° C. to obtain the final polyhedral structure.

According to the method above, the DNA tesseract is designed using Tiamat 2.0. Each of the four components (A, B, Bc, Sc) consists of four single stranded DNA occupying one face of the structure. Equimolar of single stranded DNA s (Sangon, nanodrop corrected to 10 μM) are mixed in water to make up a 2.5 μM working solution of the individual components. Equimolar of the working solutions are mixed in 1× phosphate buffer saline (Sigma; 140 mM NaCl, 3 mM KCl, 10 mM phosphate buffer) at the final concentration of 20 nM for the assembly. For gel electrophoresis, samples are prepared in 1×TAEM (40 mM Tris-acetate, 1 mM EDTA, 12.5 mM magnesium acetate). The assembly mixture is annealed in thermal cycler (Applied Biosystems ProFlex PCR System). The mixture is incubated at 95° C. for 3 minutes then slowly annealed from 95° C. to 20° C. at the rate of 4.4° C./hour. The folded structures are stored at room temperature.

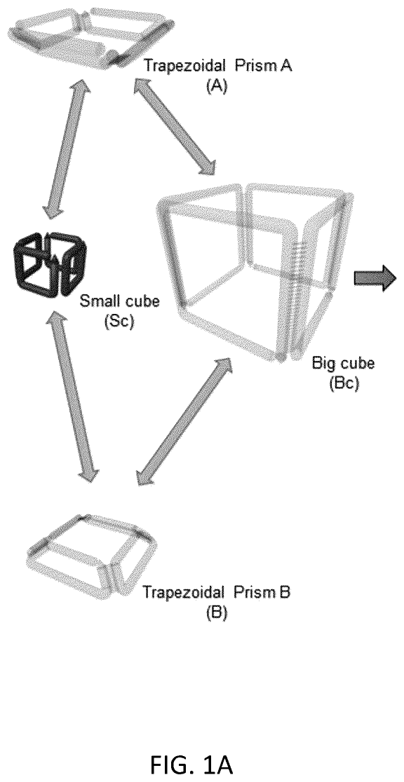

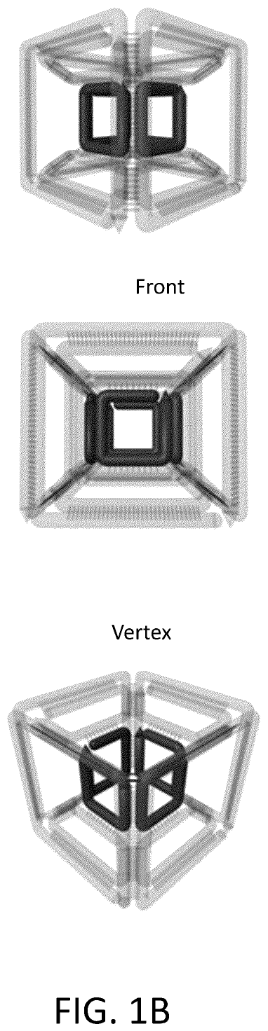

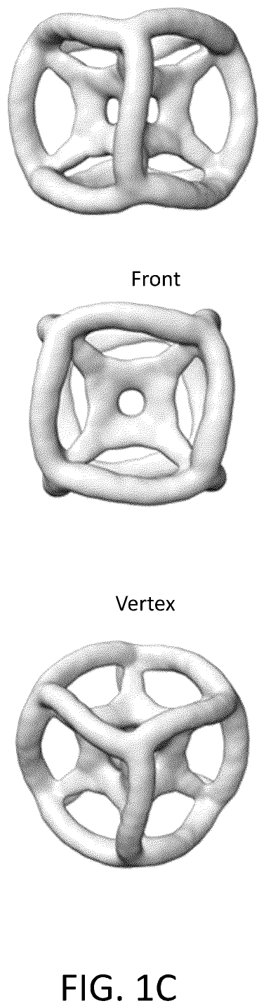

Referring to FIGS. 1A to 1C, FIG. 1A shows the components of the DNA tesseract. The shape has four sub-structures: Big cube (Bc), Small cube (Sc) and two trapezoidal prisms termed ‘A’ and ‘B’. Each sub-structure consists of four single-stranded DNAs with each strand making up one face of the sub-structure using a single thymidine at each vertex. These four sub-structures interact as depicted by the grey arrows. The small cube does not interact with the big cube directly but is captured within the tesseract through the presence of trapezoidal prisms. FIG. 1B shows various perspectives of the completed DNA tesseract at different viewing angles; and FIG. 1C shows the 3D electron density maps of the DNA tesseract determined by Cryo-EM single particle analysis, confirming the octahedral symmetry of the structure.

### 1.2 Formation of DNA Tesseract

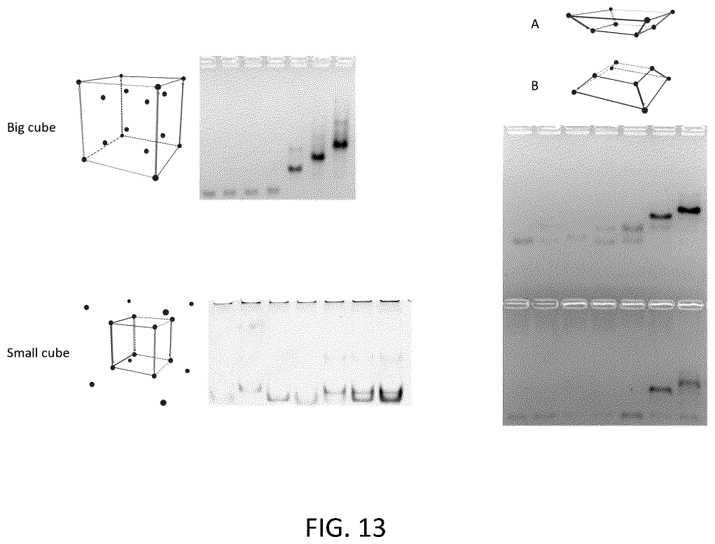

To confirm modular formation of the DNA tesseract, electrophoretic mobility shift assays (EMSAs) are conducted to evaluate the structural integrity of each sub-component. The large cube and the trapezoidal prisms form higher-order assemblies, demonstrated by a reduction in electrophoretic mobility. In contrast, the small cube does not exhibit any mobility shift, indicating an absence of higher-order assembly (see FIG. 13).

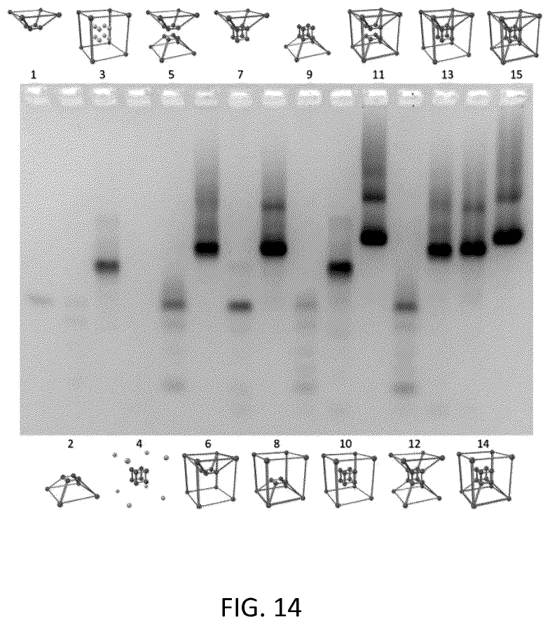

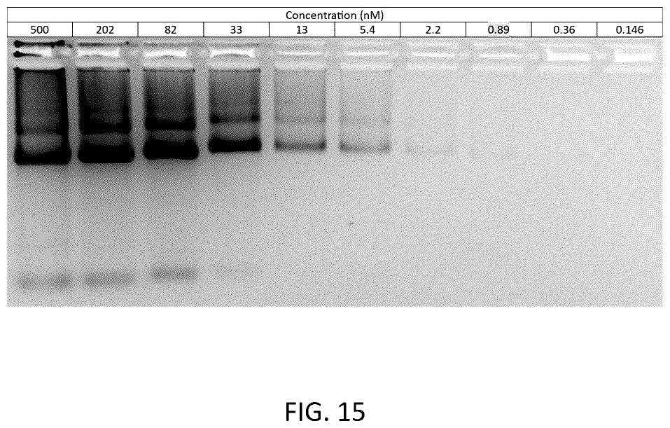

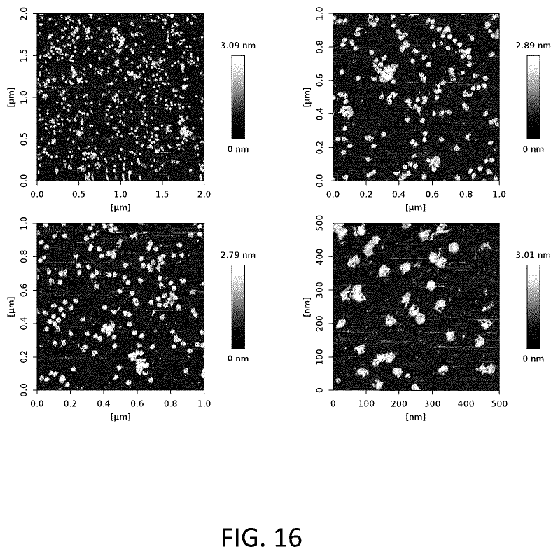

To validate that the DNA tesseract requires structural stabilization of the outer large cube by an internal small cube, all 15 possible sub-structure combinations are assembled and analyzed by EMSA (see FIG. 14). Migration retardation correlates with increasing structural complexity. Assembly efficiency of the complete tesseract is measured at 89.5% specifically by comparing band intensities of big cube on lane 3 and lane 15.

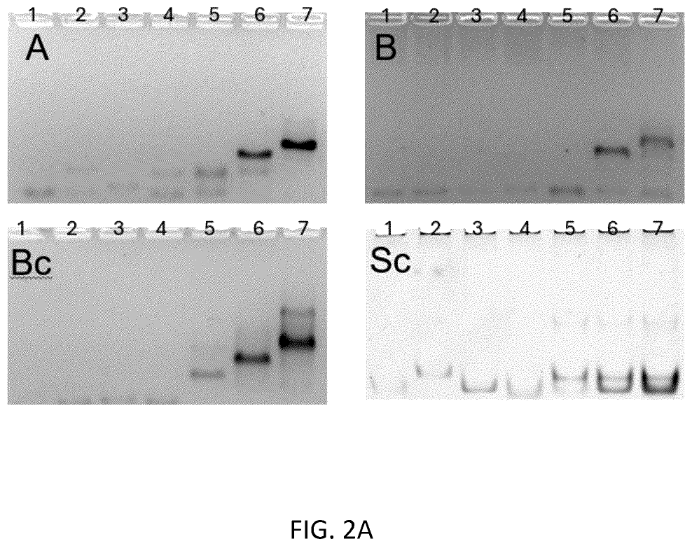

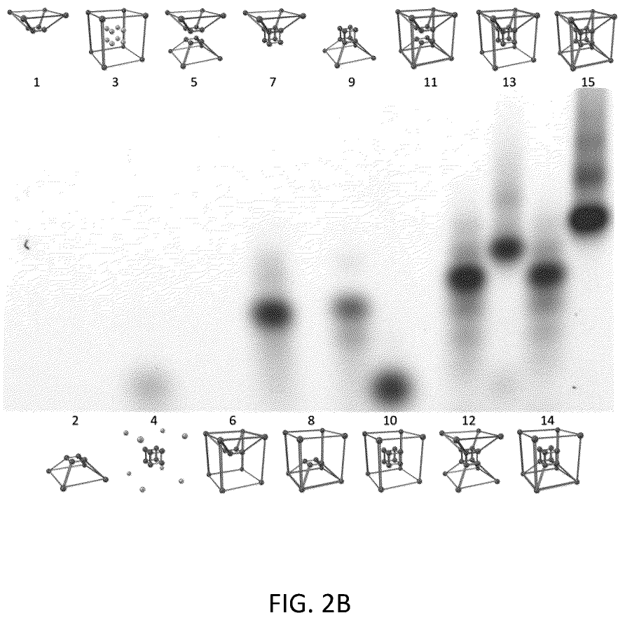

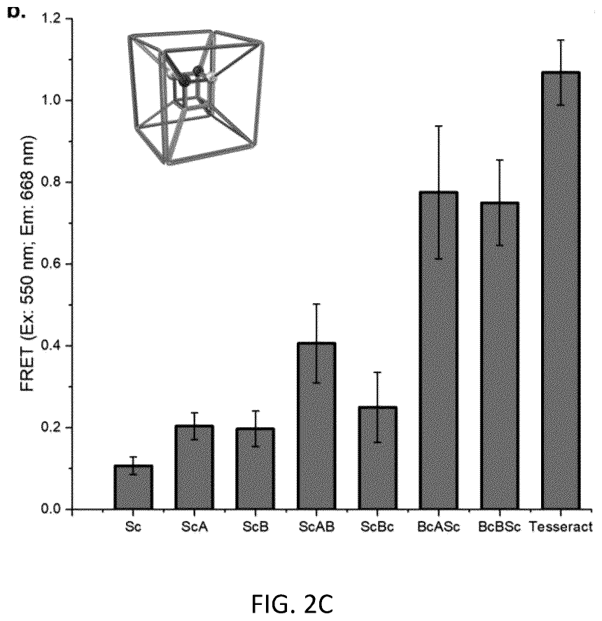

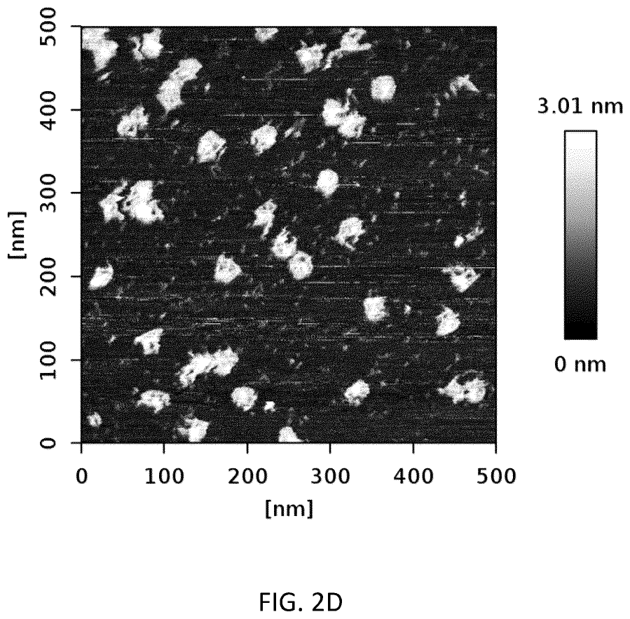

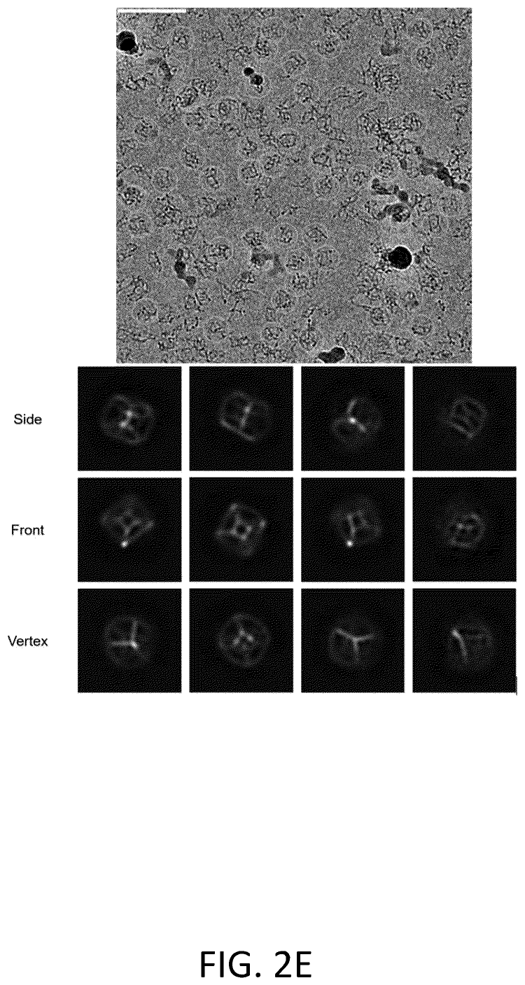

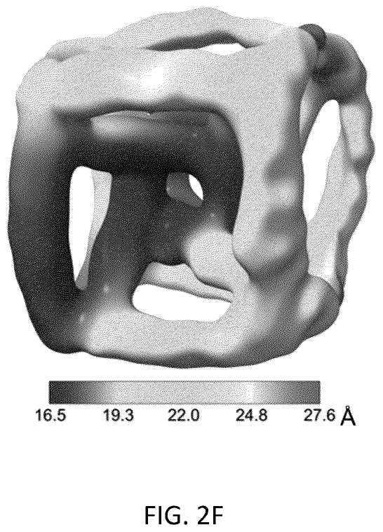

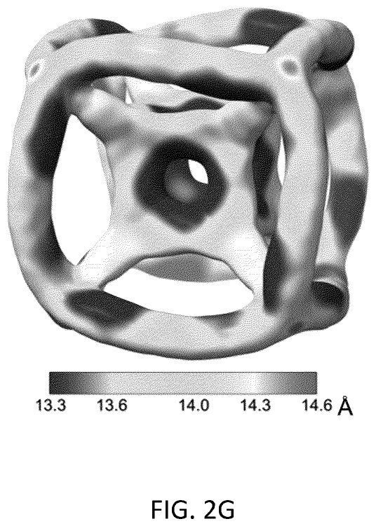

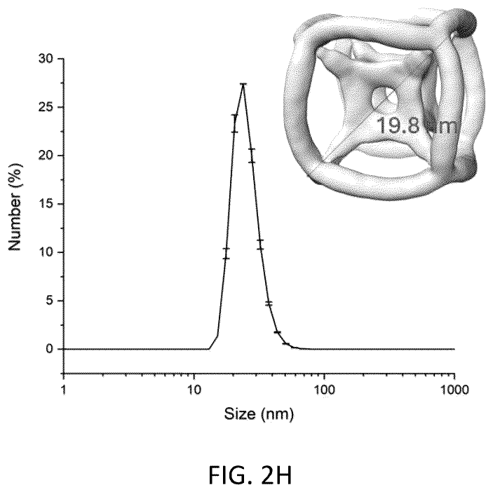

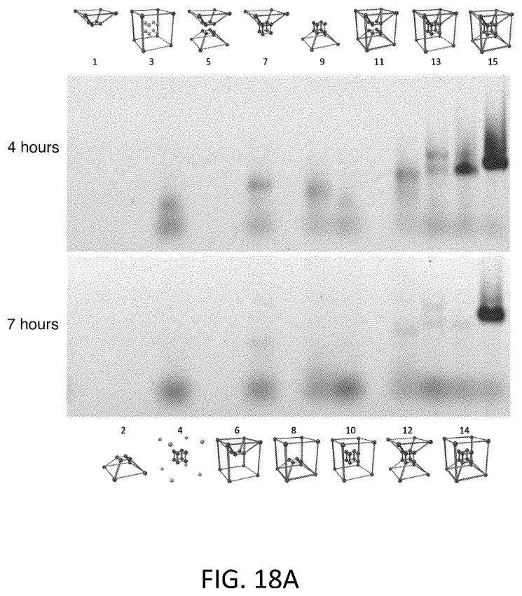

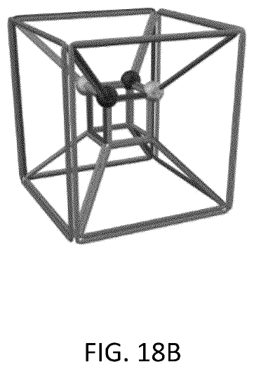

Due to the short DNA length, the small cube strands are initially undetectable. To enable detection, Cy3 and Cy5 fluorophores are attached to diagonally opposite single strands of the small cube to facilitate FRET-based fluorescence detection (see FIG. 2B in which single-stranded DNAs for different combinations of components are mixed for assembly. Lane 1: A; Lane 2: B; Lane 3: Bc; Lane 4: Sc; Lane 5: AB; Lane 6: BcA; Lane 7: SCA; Lane 8: BcB; Lane 9: ScB; Lane 10: BcSc; Lane 11: BcAB; Lane 12: ScAB; Lane 13: BcA Sc; Lane 14: BcBSc; Lane 15: BcABSc (tesseract)). Fluorescent EMSA reveals that while unassembled small cube strands yield a weak FRET signal, combinations incorporating other sub-structures exhibit significantly enhanced FRET. Notably, the complete tesseract maintains a stable FRET signal under extended electrophoresis, indicating structural robustness. See FIGS. 18A and 18B. As the FRET signal is not observed for structure 11 but observed clearly for the full tesseract in structure 15, this is an indication of stabilization of the small cube within the tesseract. The same setup is repeated with 4 hours and 7 hours of electrophoresis, where the tesseract FRET intensity remained stable after 7 hours, indicating high stability of the tesseract compared to other combinations.

To further quantify this result, FRET intensities in bulk solution are quantified. The complete tesseract yields a FRET signal tenfold greater than that of the isolated small cube, confirming that small cube assembly occurs only in the presence of the full tesseract structure.

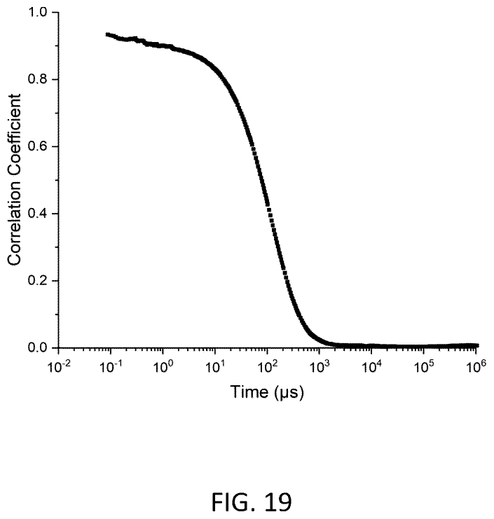

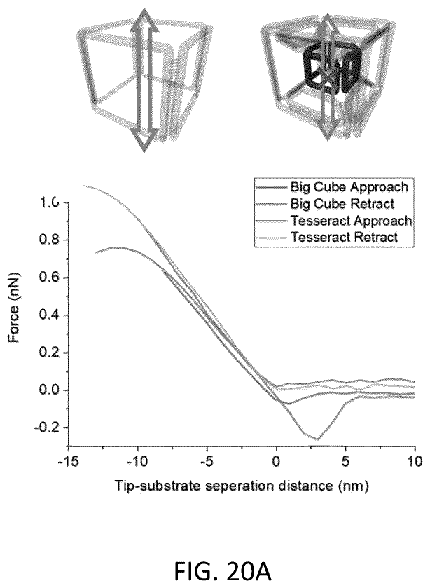

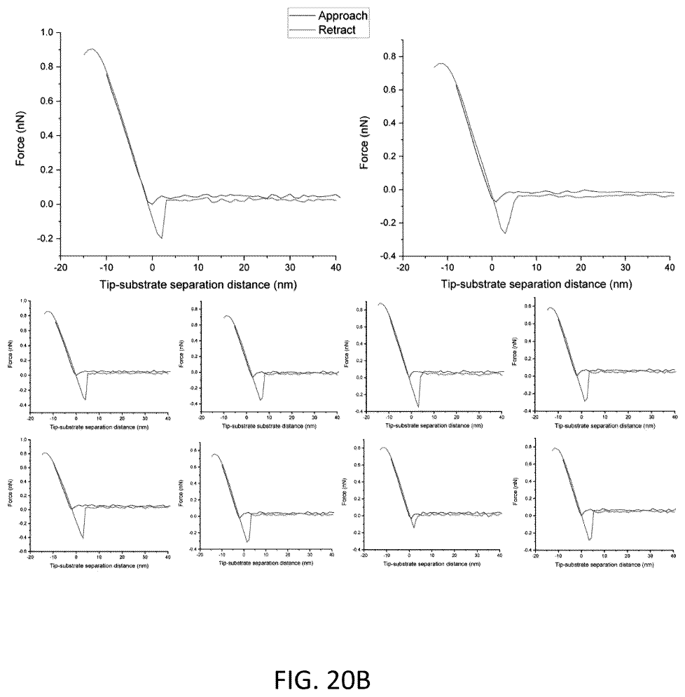

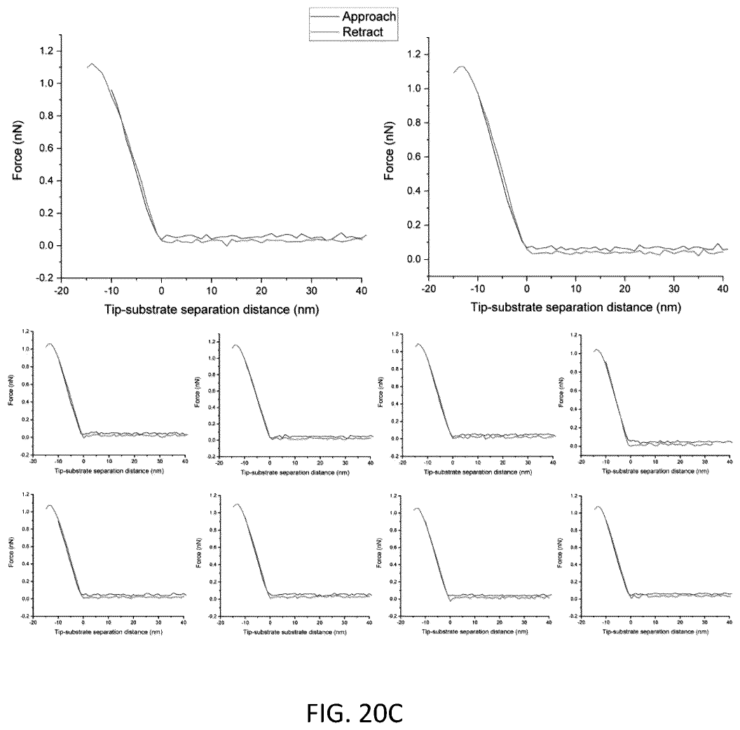

The tesseract size is subsequently characterized by dynamic light scattering (DLS) and cryo-electron microscopy (Cryo-EM), as seen in FIG. 2H. DLS determines a hydrodynamic diameter of 25.5 nm, while Cryo-EM yields a diagonal length of 19.8 nm. These measurements, corroborated by autocorrelation analysis and Cryo-EM density mapping (as seen in FIG. 19, indicated by the high intercept (>0.9), smooth and monomodal exponential decay, and a smooth baseline in the correlation function), demonstrate consistency within the expected tolerances due to formation of a double layer on particles during DLS, which results in the hydrodynamic diameter slightly greater than the particle core diameter.

### 1.3 Stability of DNA Tesseract

Atomic force microscopy (AFM) and Cryo-EM provide further structural insight. Cryo-EM reconstruction without symmetry application (C1) reveals a complete tesseract structure, where the small cube is resolved at higher resolution, suggesting increased rigidity. See FIG. 2F, in which the global resolution of C1 reconstruction is 21.24 Å; and the small cube is found to be more stable than the other parts of the structure. This observation supports its role as the structural core of the hyperstructure. Applying octahedral symmetry yields a more representative 3D reconstruction structure of the tesseract in solution by removing the fluctuation from the C1 reconstruction due to damage from freezing in Cryo-EM, as seen in FIG. 2G in which symmetry is applied to enhance the global resolution to 14.16 Å.

AFM imaging confirms even spatial distribution and a planar dimension of 10×10 nm. Force-distance spectrum of the big cube and the tesseract with AFM measuring 10 random particles on mica. Young's modulus of the tesseract is at 23.44±0.56 MPa, higher than that of the large cube (19.55±0.98 MPa) and significantly exceeding values for conventional DNA nanostructures. The modulus remains consistent during retraction evidenced by the constant slope of the force curve, indicating elastic recovery and minimal hysteresis under deformation despite the distortion of z-height of the tesseract during AFM tip approach. The energy dissipated during AFM tip retraction is 0.033±0.01×10⁻¹⁸ J for the tesseract, which is 23.7 times lower than the large cube's dissipation (0.783±0.1×10⁻¹⁸ J), indicating the internal framework resists expansion pressures and exhibits high mechanical integrity under acoustic forces. This in turn shows the applicability of the DNA tesseract of the present invention in high frequency surface acoustic wave (SAW) applications.

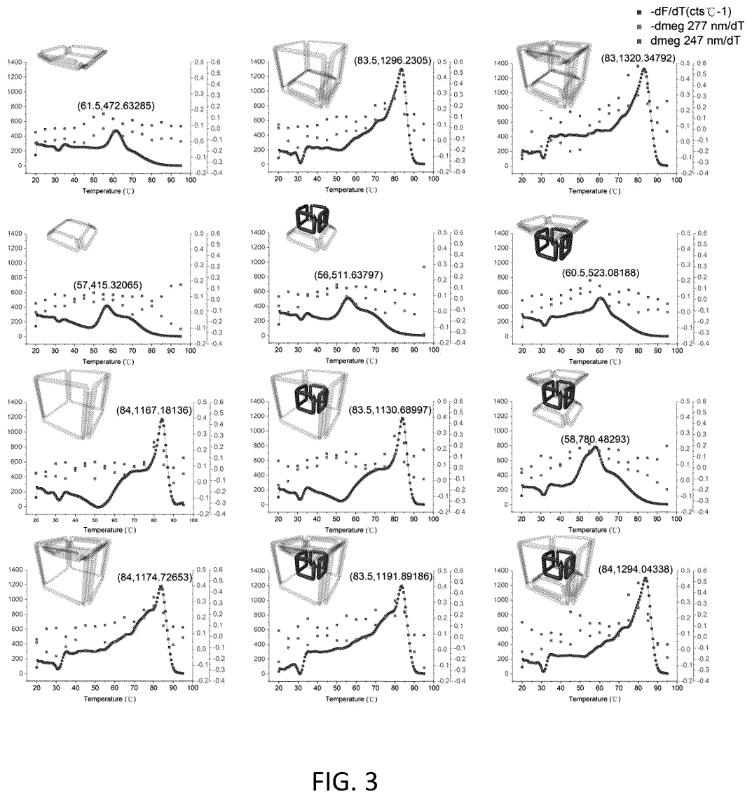

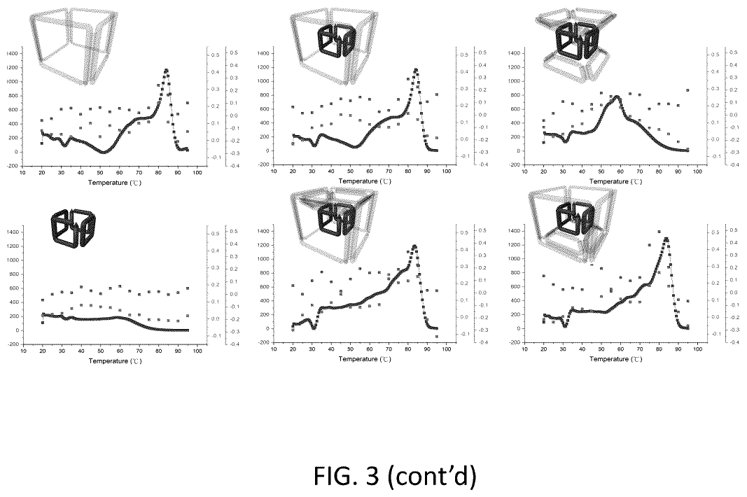

Thermal stability is assessed using real-time qPCR and circular dichroism (CD), as seen in the melting profiles in FIG. 3. Sub-structure combinations exhibit melting temperatures (Tₘ) that scale with structural complexity, as seen in real-time qPCR results. The full tesseract achieves a Tₘ of 83.5-84° C., confirmed by CD signal retention at 247 and 277 nm as signature peaks of duplex NDA. When concentrations of small cube and complete tesseract are raised to 1.7 μM, qPCR detects a 54° C. Tₘ for the small cube, with no corresponding peak in the tesseract sample, indicating the high thermal stability of the complete structure.

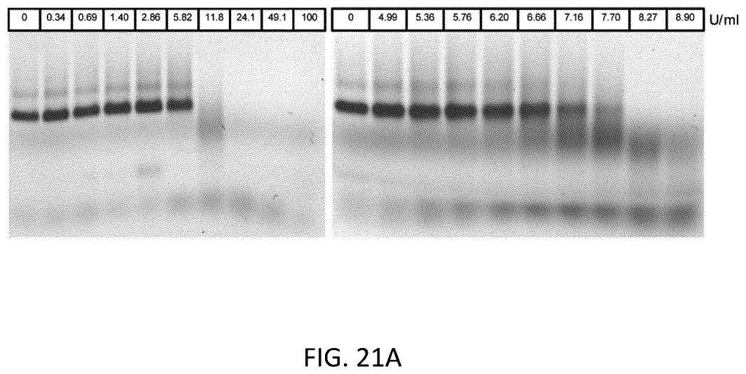

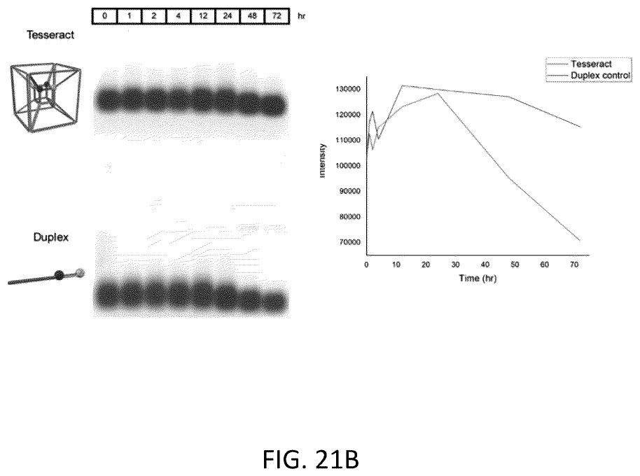

Nuclease resistance is evaluated using DNase-I digestion and fetal bovine serum incubation, seen in FIGS. 21A and 21B. The tesseract resists degradation until DNase-I concentrations reach 7.7 U/mL, 21-fold higher than physiological levels. Incubation in 95% fetal bovine serum for three days results in no significant structural degradation, confirming the tesseract's stability in biologically relevant conditions.

1.4 Alignment of DNA Tesseract into Conducting Wire

### 1.4.1 Theory of Assembly Driven by Standing SAW

Tesseract clusters assemble into fibers driven by an acoustic force (F^{rad}) provided by SAW field, as described by following equation:

$${F}^{rad} = 4 πΦ {ka}^{3} {E}_{ac} sin ( 2 kx )$$

where k=2π/λ is acoustic wavenumber, a is the radius of DNA clusters, E_{ac} is acoustic energy density, x is the position of clusters along the direction of SAW propagation, Φ is acoustophoretic contrast factor described by:

$$Φ = \frac{1}{3} ( \frac{5 {ρ}_{p} - 2 {ρ}_{0}}{2 {ρ}_{p} + {ρ}_{0}} - \frac{{β}_{p}}{{β}_{0}} )$$

where ρₚ and ρ₀ are mass densities of DNA clusters and water respectively, βₚ and β₀ are their respective compressibility. If Φ is positive, DNA aggregates tend to move toward the pressure nodes, otherwise they will be driven to the antinodes.

As DNA clusters move in water driven by the acoustic radiation force, they will be subject to the friction force from the water named drag force (F^{drag}):

$${F}^{drag} = 6 πη a ( 〈 {v}_{2} 〉 - {v}_{p} )$$

where η is the dynamic viscosity of water, (v₂) is second-order time average velocity from acoustic streaming, and vₚ is the velocity of DNA clusters. Acoustic streaming is strongly related to the boundary layer in a confined channel and thermal inhomogeneity in the liquid. In the setup of the present invention, the wavelength is much smaller than sizes of the capillary used for holding DNA on the surface of a SAW devices as well as heat accumulation is strictly limited by using pulse signal input, therefore reducing acoustic streaming drastically.

The motion of DNA clusters is determined by the relative magnitude of F^{rad} and F^{drag}. The success of DNA assembly can be achieved when F^{rad} is larger than F^{drag}. Reducing acoustic streaming contributes to a small value of drag force. At the meantime, acoustic radiation force is enhanced by increasing the size of DNA cluster since F^{rad}/F^{drag} is proportional to the square of DNA cluster size. It is observed that the clusters of DNA tesseract with the size smaller than the distance between two adjacent nodes standing SAW under an optical microscope and SAM, indicating F^{rad} is dominant enough to assemble DNA clusters.

### 1.4.2 Fabrication of the Tesseract Wire

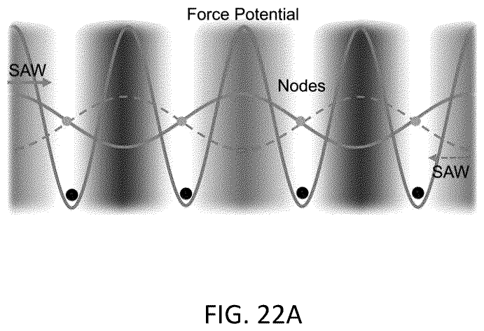

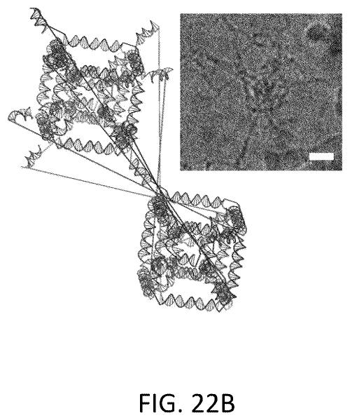

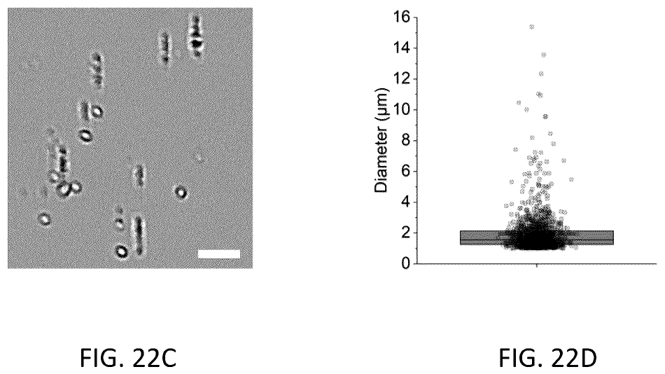

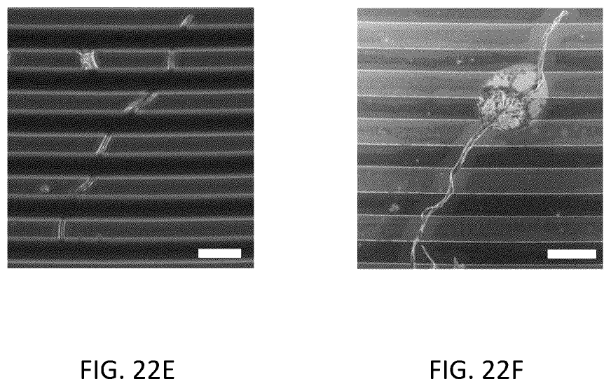

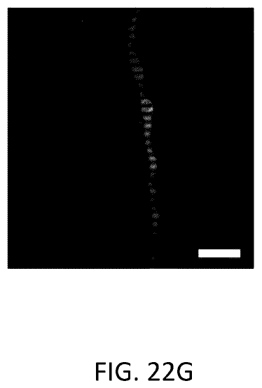

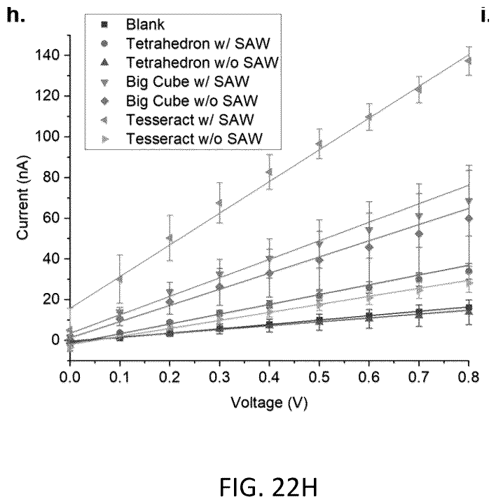

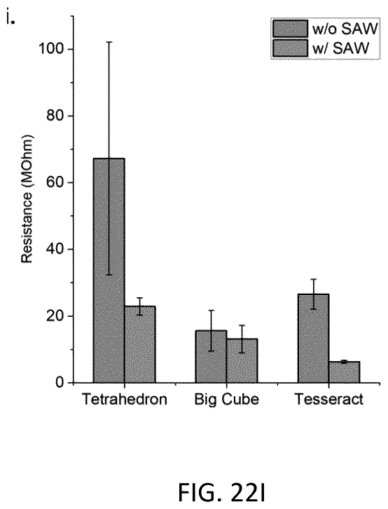

To explore scalable fabrication, surface acoustic wave (SAW) alignment is employed to assemble tesseract clusters into sub-millimetre wire-like structures as illustrated in FIG. 22A.

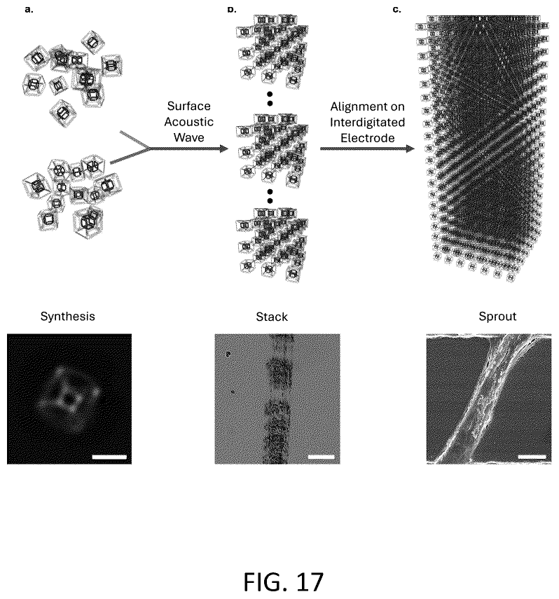

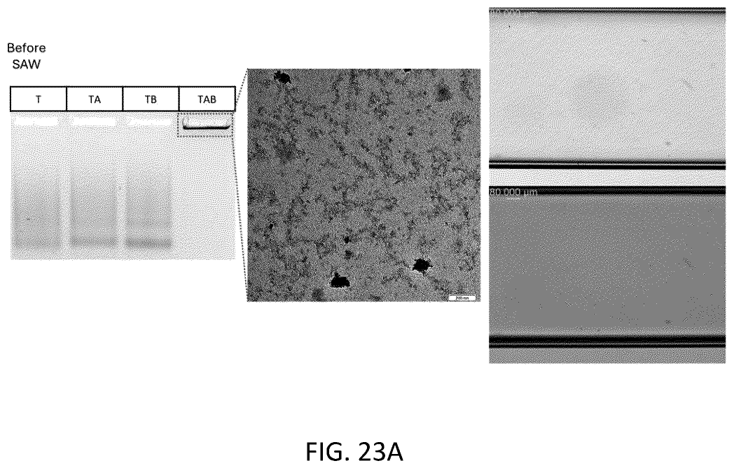

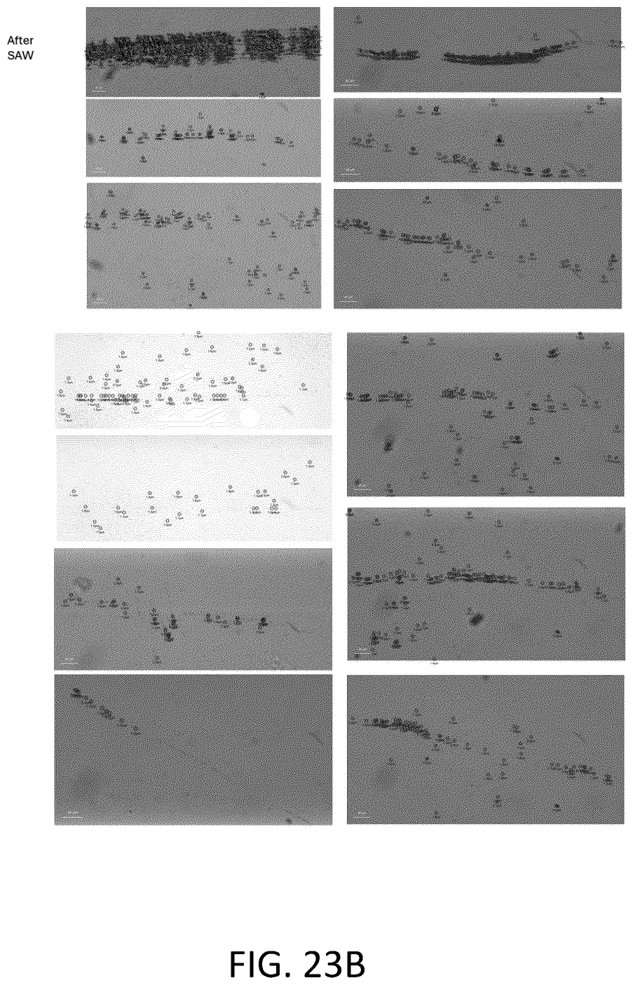

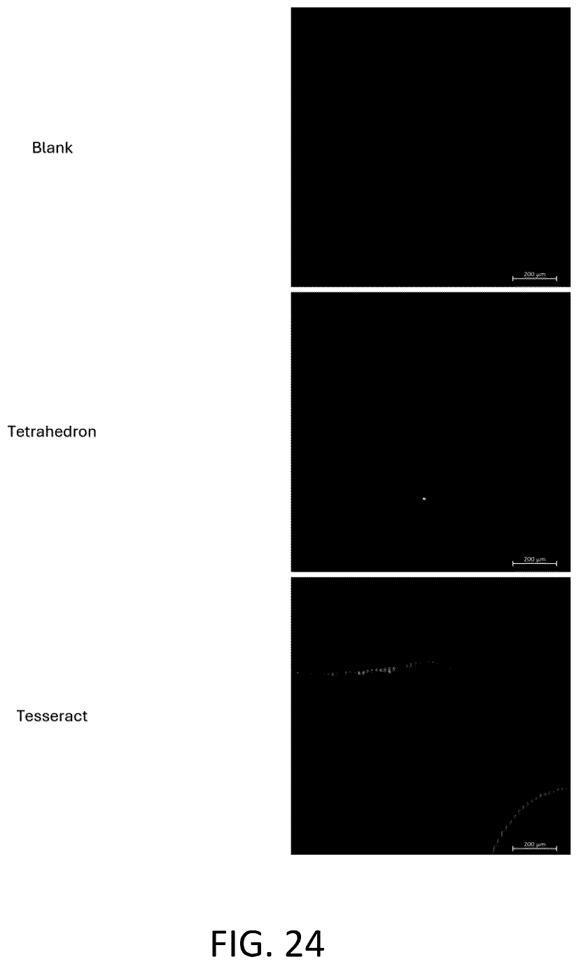

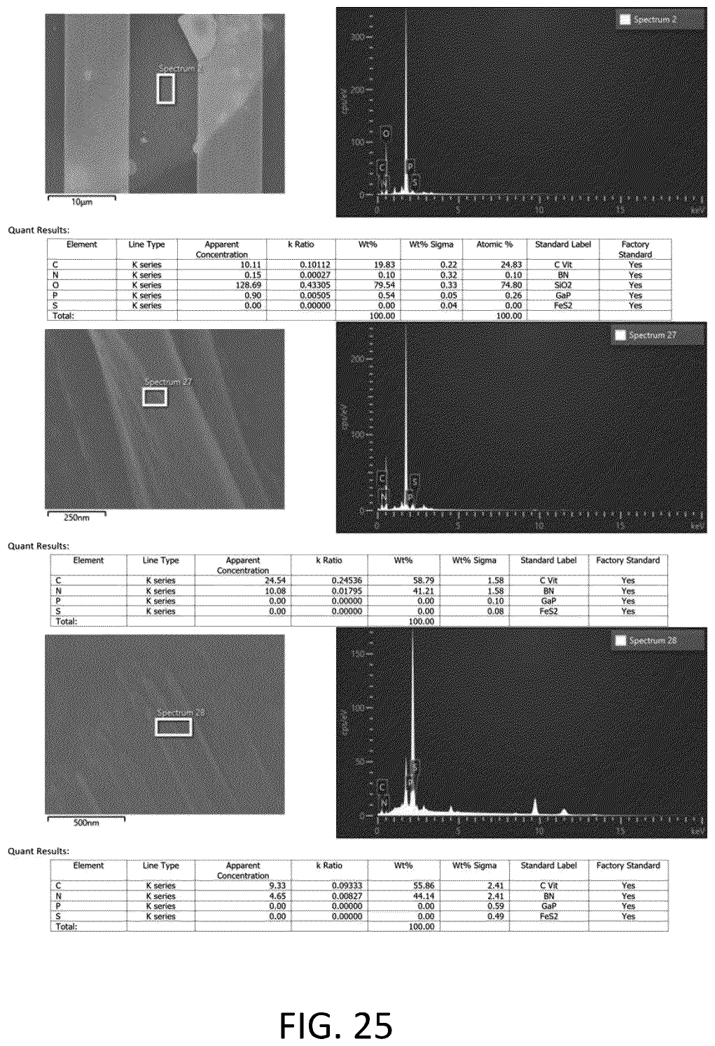

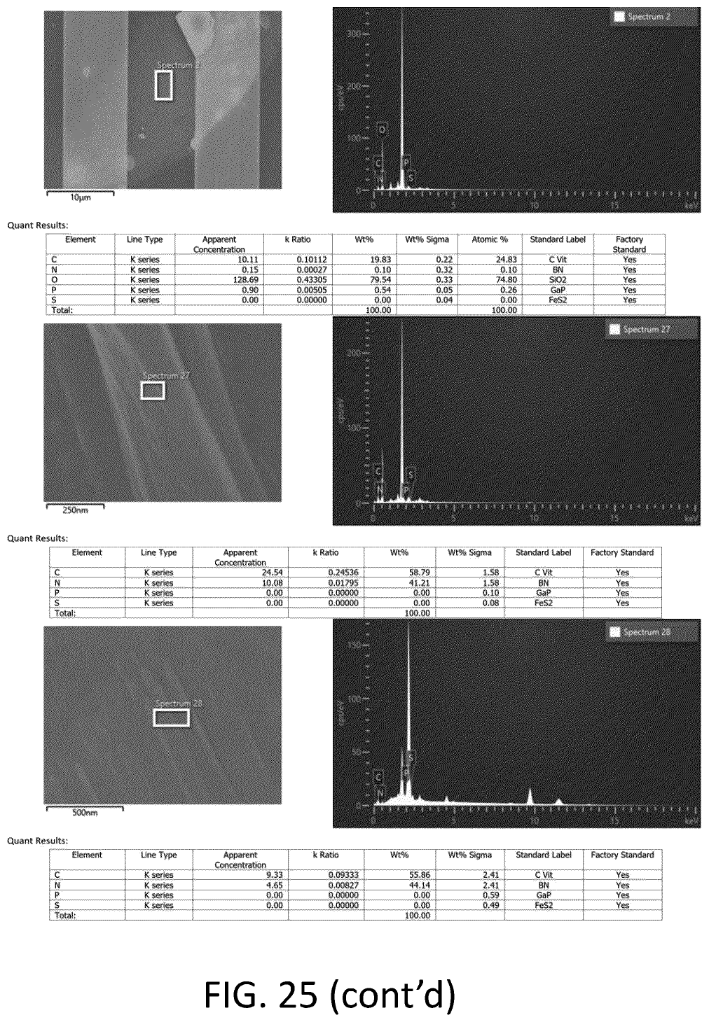

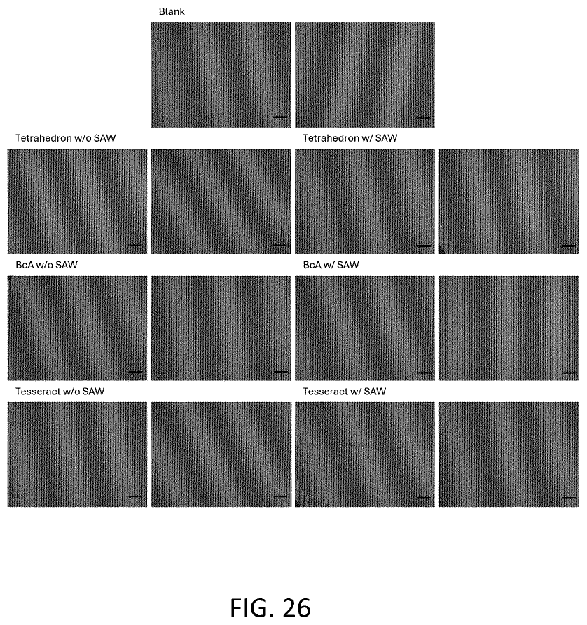

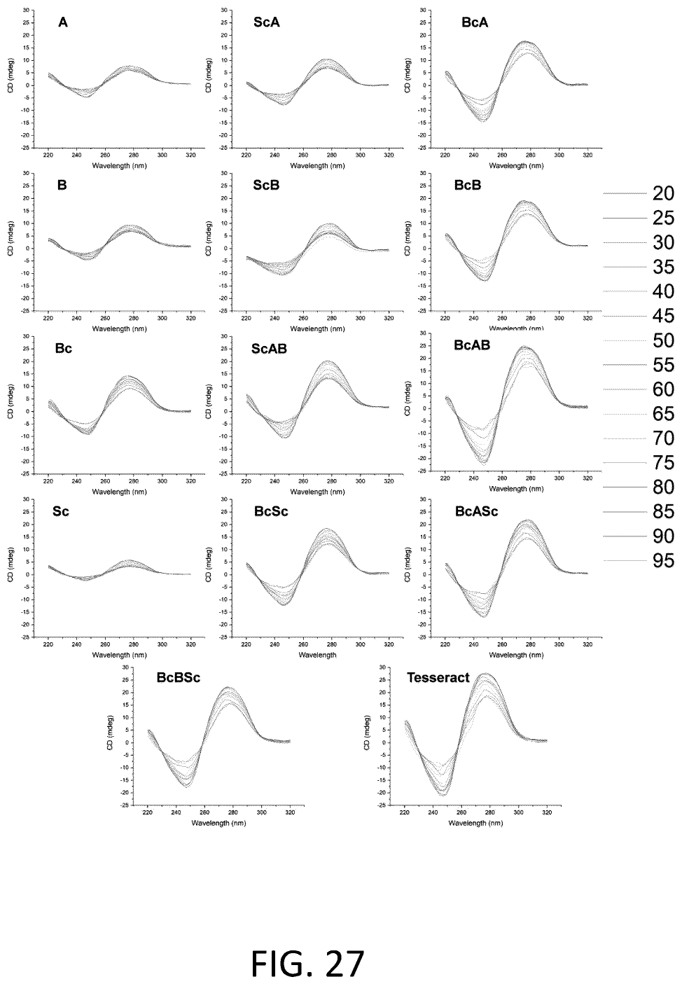

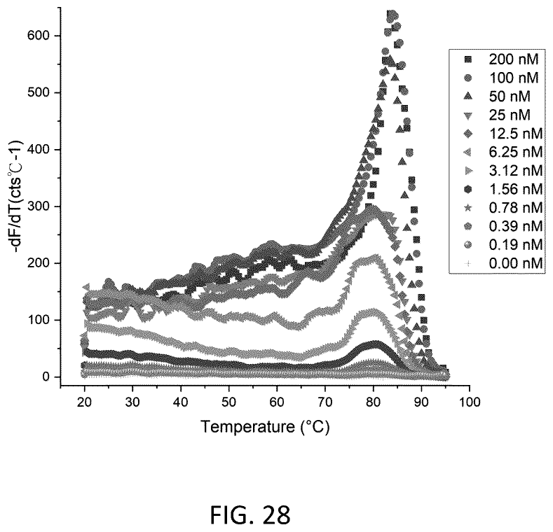

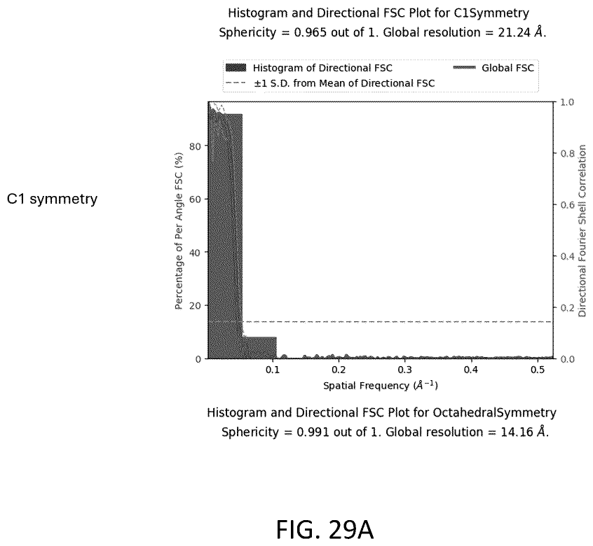

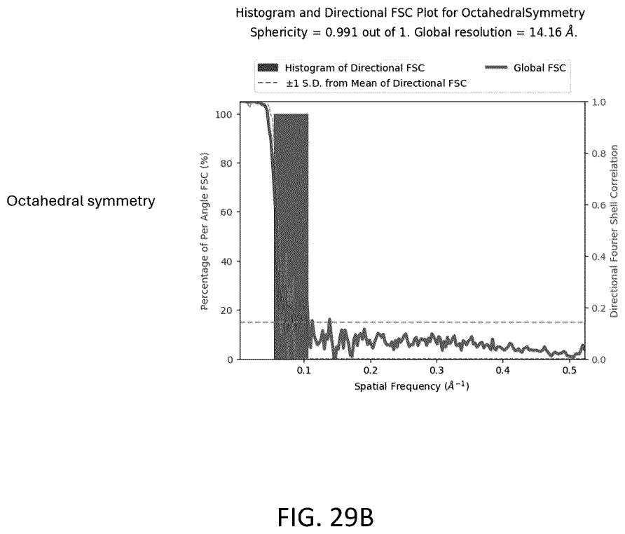

The strategy of bottom-up assembly of a DNA tesseract wire is illustrated in FIG. 17. Eight duplexes as overhang sequences are designed using Tiamat, as seen in FIG. 22B, to promote diagonal linkage between tesseracts. Equimolar compositions of modified tesseracts, separately assembled and purified via Cryo-EM respectively, are mixed to trigger hybridization-driven aggregation (FIG. 23A), and introduced into a thin rectangular capillary chamber positioned between interdigital transducers (IDTs) on a piezoelectric substrate. A standing SAW generated by IDTs is applied across the capillary to align the tesseract aggregate into a stream of large clusters (see FIGS. 22C and 23B). The diameter of clusters identified from the capillary are measured to have average of 1.93 μm and median of 1.55 μm, evidenced in FIG. 22D. The liquid is pushed out of the capillary and deposited on an interdigitated electrode on which thiolated anchors are immobilized for capturing the wire via duplex hybridization with ssDNA overhangs. Continuous tesseract wires exceeding 100 μm in length are thus yielded, as evidenced by brightfield microscopy (see FIGS. 22E and 23B) and scanning electron microscopy (see FIG. 22F). A Cy3 anchor is further used to stain the electrode and signal is observed under confocal microscopy, as shown in FIGS. 22G and 24. Energy-dispersive X-ray spectroscopy in SEM also detected presence of phosphorus and nitrogen, indicative of the presence of DNA (see FIG. 25).

Electrical conductivity of the wires is assessed using a potentiostat. Application of a linear voltage across interdigitated electrodes (IDEs) indicates enhanced current and reduced resistance after SAW alignment, as seen in FIG. 22H. Structural variants, including an incomplete tesseract (BcA) and a tetrahedron with equivalent edge length, fail to form conductive wires and exhibit higher electrical resistance as compared to the completed tesseract wire (see FIG. 22I), indicating the structural stability and scalability of the DNA tesseract of the present invention beyond DNA origami, having immense potential in large-scaled DNA-based bioelectronics.

### Example 2—Materials and Methods

### 2.1 Gel Electrophoresis

A garose gel electrophoresis is conducted using 2.5% agarose gel in 1X TAEM to observe the migration of DNA tesseract and the partial assemblies. Polyacrylamide gel electrophoresis (PAGE) is only used to observe the assembly of small cubes; specifically, 15% native PAGE in 1×TAEM is used. Gel electrophoresis is performed at 65 V at 4° C., with 4 hours for agarose gel electrophoresis and 1 hour for the PAGE. Fluorescent signasl from the agarose gel electrophoresis is observed with Analytical GE Amersham Typhoon5 Biomolecular Imager (photomultiplier tube potential: 322 V). Sybr gold (Invitrogen)-stained samples are pre-stainede by mixing 10 μl of 20 nM sample with 2 μl loading mixture (5× Sybr gold, 4× loading dye). Signal is observed with chemidoc (Bio-Rad).

### 2.2 Quantitative Fluorescent Measurement

20 nM of DNA tesseract and partial assemblies are assembled in 1×PBS (strands on small cube are labeled with Cy3 and Cy5). The fluorescent signals of fluorescent resonance energy transfer (excitation: 550 nm, emission: 668 nm) are measured using Thermo Varioskan Flash microplate reader. Bandwidth is 5 nm and measurement time is 500 ms.

2.3 Quantitative Polymerase Chain Reaction (qPCR)

In qPCR, 100 nM of DNA tesseract and partial assemblies are assembled in 1×PBS. Sybr green is used to the observed the presence duplex DNA in the mixture. The samples are subjected to melting from 20° C. to 95° C. at the rate of 0.5° C./min in Biorad Opus 96. The derivatives of raw data are calculated to find out the melting temperature.

### 2.4 Circular Dichroism (CD)

300 nM of DNA tesseract and partial assemblies are assembled for CD using Jasco J-1500. 600 μl of sample is transferred to a quartz cuvette with 1 cm pathlength. Five repetitive measurements are done by scanning the spectrum from 220 nm to 320 nm with 1 nm bandwidth. Data pitch is 1 nm and data integration time are 2 seconds. In order to observe melting, measurement is repeated for every 5° C. from 20° C. to 95° C. The peak values from 247 nm and 277 nm are obtained to calculate the derivatives along the temperature change.

### 2.5 Dynamic Light Scattering (DLS)

Particle size and size distribution of the DNA tesseract are characterised by dynamic light scattering (DLS), using the Zetasizer Pro particle size analyzer with a 4.0 mW He—Ne laser at a detection angle of 173°, and the ZSX plorer software (Malvern Panalytical Ltd., Worcestershire, UK). Samples are dispersed in 1×PBS, loaded in a low-volume quartz cuvette and measured in triplicates.

### 2.6 Atomic Force Microscopy

AFM imaging is performed using a Bruker NanoWizard ULTRA Speed 2 A FM, employing the peakforce tapping mode in a fluidic environment. SNL-10d AFM tapping mode probes (Bruker Nano, Inc.) with an average force constant of 0.06 N/m and a mean resonance frequency of 18 kHz is used in imaging with scan areas of 0.5 μm×0.5 μm. A scanning resolution of 256 lines with 256 pixels per line with a scan rate of 1 Hz is maintained throughout all image acquisitions. The JPK Data Processing software program is utilized for the meticulous analysis of DNA contour lengths, as previously described.

The AFM and force-distance curve measurements are carried out in 1×TAE buffer with 7 mM NiCl₂ and analysed following a publwashed protocol. To obtain Young's modulus (E) value, the deflection sensitivity of the probe is calibrated on the mica surface several times and the mean value is taken. The spring constant of the probe is determined using the Thermal Tune function of the AFM software. The Young's modulus (E) value is determined by fitting the data to the Sneddon (conical indenter) model, where F=force (from force curve), E=Young's modulus (fit parameter), v=Poisson's ratio (sample dependent, typically 0.2-0.5, for the DNA Tesseract, the Poisson coefficient should be around 0.3), α=half-angle of the indenter (20 degree), δ=indentation.

### 2.7 Cryo-EM Sample Preparation, Data Collection and Analysis

The assembled DNA tesseract at 20 nM in PBS is prepared in sufficient amount to be concentrated using A micron centrifugal filter with 100 kDa cutoff. Centrifugation is performed at 140,000 g for 10 minutes at room temperature. Multiple centrifugations are performed until the concentration of DNA tesseract reached 10 μM as confirmed by nanodrop. 3.5 μl of concentrated DNA Tesseract is then applied to glow-discharged holey carbon gird (Quantifoil 1.2/1.3) and vitrified using Vitrobot mark IV (ThermoFisher Scientific) at 4° C., 100% humidity, 0 s wait time, 3 s blot time and 0 blot force. The DNA tesseract is imaged at nominal magnification of 130,000× on a 300 KV FEI Titan Krios, with a pixel size of 0.9557 Å. Movies are captured in counting mode with electron dose rate at 50 eÅ⁻² using the EPU software and Falcon 3 detector (FEI) with a defocus range of −1.2 to −2.6 μm. The raw movies are motion corrected, followed by constant transfer function (CTF) estimation in CryoSPARC 4.4. Particles are manually picked as template for Topaz training to generate a picking model. The model is used to automatically pick particles from the entire movies set. A sub-set of the picks is inspected and corrected as new template for another round of training. Three Topaz training is performed in the same manner for auto-picking the dataset. 2D and 3D classification is performed to select intact particle for the reconstruction. 4,745 particles are selected to reconstruct a 3D map using the ab initio model with C1 symmetry. To enhance the resolution of the 3D map, octahedral symmetry is applied for the reconstruction again as well as the non-uniform refinement. Half-maps and full map are submitted to 3DFSC processing server Remote 3DFSC Processing Server (salk.edu) for validating the final resolution. The illustrations of refined maps and 3D resolution maps are performed in ChimeraX.

### 2.8 Surface Acoustic Wave (SAW) for DNA Tesseract Alignment

DNA tesseract, BcA or tetrahedron with two different set of complementary overhangs are assembled separately at 20 nM in 1×PBS. The products are concentrated to 200 nM and mixed for another slow annealing from 50 to 20° C. over two days. Samples are then transferred into hollow rectangle capillaries (CM Scientific, 0.05 mm×1.00 mm×50 mm) through capillary action and sealed with nail polish.

### 2.9 SAW Device Fabrication

Interdigital transducers (IDTs) are patterned on a Y-cut lithium niobate (LiNbO₃) substrate via photolithography, with a 1500 μm gap between transmitter and receiver IDTs. Each IDT comprised 40 finger pairs of titanium/aluminum electrodes (2.5 μm width, 210 nm thickness), generating SAWs with a 10 μm wavelength at 342 MHz. The IDTs are wire-bonded to a printed circuit board (PCB).

### 2.10 Acoustic Alignment and Deposition

Clustered nanostructures are loaded into rectangular glass capillaries (0.05×1.00×50 mm, CM Scientific) via capillary action, and both ends are sealed with UV-curable epoxy. The capillary is mounted between the IDTs on the LiNbO₃ substrate, with a thin layer of immersion oil (Nikon, Type A) applied to enhance acoustic coupling. A sinusoidal signal (342 MHz, 2 V pp) is delivered to the transmitter IDT using a function generator (Siglent SDG 7102A), pulsed at 3 ms duration (1,000,000 cycles) with 27 ms intervals to mitigate thermal drift. Standing waves formed via interference between incident and reflected waves, concentrating nanostructures at pressure nodes within 30 minutes.

After applying SAW, one end of the capillary is cut, inserted into a cut gel loading tip and sealed with polydimethylsiloxane (PDMS). After the PDMS is dried, the other end of the capillary is cut, and the solution is pushed with a pipette.

### 2.11 Electrode Biofunctionalization and Tesseract Wire Hybridization

MicruX interdigitated gold electrodes (ED-IDE 1-Au) are first rinsed thoroughly with 95% ethanol and MiliQ water. After drying, 10 μl of 0.5 M H₂SO₄ is dropped onto the electrode. The gold surface is activated by cyclic voltammetry (CV) scanning from −1 V to 1.3 V at 0.1 V/s for 12 cycles. The electrodes are rinsed again by MiliQ water and dried before DNA immobilization.

Thiol modified DNA anchor strands are reconstituted to 100 μM and diluted to 10 μM in ultra-pure water. Then, the strands are reduced by Tris-(2-Carboxyethyl) phosphine (TCEP; oligo to TCEP molar ratio is 1:100) for 2 hours at room temperature (RT) to cleave the disulfide bonds. Reduced anchor strands are mixed and diluted to 0.5 μM each in 1×PBS. 10 μl of the mixture is dropped onto the activated gold surface and immobilized at RT overnight. After thorough washing with MiliQ water, the electrodes are blocked with 6 mM of mercaptohexanol (MCH) for 3 hours at RT. Following the washing off unbound MCH, 5 μl of sample is dropped onto the gold surface for strand hybridization. The reaction is performed at RT overnight. All incubation steps are conducted with humidity control to prevent solution from drying.

### 2.12 Electrical Conductivity Measurement

After hybridization, the electrodes are rinsed briefly with 1×PBS and then blown dry. The presence of wire on IDE was first visually confirmed by inverted microscope. Linear sweep voltammetry (LSV) is performed using PalmSens 4 potentiostat (Houten, Neterlands). The LSV scan ranged from 0 V to 0.8 V at a rate of 1 V/s. Electrical current readings are recorded at 0.1 V intervals. All electrochemical data were exported from the software PST race 5.9.

### 2.13 Scanning Electron Microscopy (SEM)

SEM is performed using Hitachi S-4800 field emission scanning electron microscope equips with a cold cathode field emission column emitter. The IDE with tesseract wire is sputter-coated with a 60:40 gold/palladium target using Quorum Q150T Plus ES for 40 seconds. Images are obtained with secondary electron at 5 kV accelerating voltage and sample distance of 14.2 mm. For energy dispersive X-ray spectroscopy, secondary electron at 20 kV and a X-Max 80 EDS detector ae used with at least 150000 counts Reports of element mapping were generated by A Ztec software.

### 2.14 Confocal Microscopy

IDEs with observable wires under brightfield microscope were stained with 10 μl of 1 μM Cy3 anchor for 15 mins. Fluorescence imaging is performed using a Zeiss LSM 900 inverted confocal microscope equipped with A iryscan 2. Imaging is conducted with a 20× objective, with excitation and emission at 548 nm and 561 nm (detection wavelength 569 nm-617 nm) respectively. GaA sp-Pmt2 imaging device with GaAsP-PMT detector was used at 800 V detector gain. Effective numerical aperture was 0.8 and pinhole size was 122 μm. All images are processed using Zeiss ZEN Blue software for Airyscan reconstruction with only linear adjustments applied to brightness and contrast to preserve data integrity.

### 2.15 Image Analysis and High-Density Particle Characterization

Automated image processing was implemented in MATLAB (R2024b) to quantify high-density particle dimensions from micrographs acquired under brightfield microscope. The analytical workflow comprised of five stages:
(i) Interactive ROI Selection: Operators could either delineate rectangular ROIs using the draw rectangle tool or opt for full-frame processing of 16-bit TIFF images (4000×3000 pixels). Selected spatial coordinates (x, y, width, height) were logged to ensure positional reproducibility across repeated analyses.
(ii) Adaptive Preprocessing: Raw images underwent grayscale conversion followed by dual Gaussian filtering: Primary filtration (σ=2 kernel) suppressed high-frequency noise while preserving macroscale features; Secondary filtration (σ=1.5 kernel) enhanced edge contrast in low-intensity regions. This cascaded approach balanced noise reduction with structural preservation, particularly for sub-10 μm particles.
(iii) Threshold-Based Segmentation: Particle-containing regions were isolated through dynamic intensity thresholding, initiated at a base threshold of 70 (8-bit intensity scale) and adaptively calibrated within a range of 70-100 to account for variations in image contrast and illumination. Threshold values were optimized per image through visual verification, ensuring maximal inclusion of true particles while systematically excluding diffraction-limited artifacts (<1 μm). Binary masks, generated via the operation bwImg=filteredImg<darkThreshold, were rigorously validated against raw images to confirm segmentation accuracy and minimize false positives.
(iv) Morphometric Quantification: The region props algorithm extracted geometric parameters, including centroid coordinates, projected areas, and equivalent diameters, with spatial calibration derived from scale bar measurements (40-μm scale: 200 pixels=0.20 μm/px; 20-μm scale: 250 pixels=0.08 μm/px).

An image recognition mode implemented dynamic scale calibration (pixel/μm conversion) and outlier rejection to ensure measurement fidelity. All analyses preserved spatial resolution of original TIFF images (16-bit depth, 2048×2048 pixels). Validation tests confirmed <5% diameter measurement variance compared to manual tracing (n=300 particles).

### 2.16 Exemplary List of Oligonucleotides for DNA Tesseract Formation

Table 1 below shows three sets of 16 ssDNA oligonucleotides applicable for formation of DNA tesseracts.

TABLE 1

| | SEQ | |
|---|---|---|
| | ID | |
| Set | NO: | Oligonucleotide sequence |
| 1 | 1 | TAGGGCGCCCGTTAACCATGGCGCCGAGCGATGGGCAAAAAAGCCTGTTCCCTCATGAATGGGAACGGT |
| | 2 | GTTGGAACGGTCGTGGCACGCGTCGCCGTCATTCGCTCGGCGCCATGGTTAACGGGCGCCCTATGGGAG |
| | | GCGGTGCCGTCACATGCCCTGCTCCTTGGTTAGGCCCGAATTTCGGCATGTCACGCGG |
| | 3 | TGCCGAGGAATGAGCGCAGGTGTCGAACACTTGCTGCGCTCTAATTTCCGGAGTTAGCGCCACTTGACG |
| | | GCGACGCGTGCCACGACCGTTCCAACTTTTGTGAGCTAAAACGGCCCGGGCTTGCATC |
| | 4 | ACGGGCCGGACTCTCCGCGTGACATGCCGAAATTCGGGCCTAACCTGGGCCGGTGCCCTTCGCATTTGG |
| | 5 | AGGAGCAGGGCATGTGACGGCACCGCCTCCCTAGGGAAAGCCTGTGTACGTTACAGTCATCTGTGACCA |
| | | CACGATTCTGTGCTGGTCCCGGGCCGTGGCACATACCGACAAGCCATACCGCGCCACG |
| | 6 | CGGCCCGGGACCAGCACAGAATCGTGTGGTCTGGATACTAATCGGTCAGGTGGGCCCGAGTGATAGTGT |
| | | TCGACACCTGCGCTCATTCCTCGGCATCTTCAAAATTGGGCTTGAAGCTAGCTGTGTG |
| | 7 | TCACTCGGGCCCACCTGACCGATTAGTATCCTAGGAGAGCCGGTGTGAAGGCCAGTCACGGCGCCGTAA |
| | 8 | ACGTCGGCGTGGCTCACACAGCTAGCTTCAAGCCCAATTTTGAAGTACGATAGTAGAGCTATCCGTGCT |
| | 9 | CAGATGACTGTAACGTACACAGGCTTTCCCTTACCGTTCCCATTCTCCAGGCTGGTCACCGGCTCTCCT |
| | 10 | AGGGCACCGGCCCTCGTGGCGCGGTATGGCTTGTCGGTATGTGCCTGCCACGCCGACGTTCGCTCTCCT |
| | 11 | GTGGCGCTAACTCCGGAAATTAGAGCGCAGCTTTACGGCGCCGTGTGGAGGGTGTTGGCTTTTTTGCCC |
| | 12 | GCTCTACTATCGTTGATGCAAGCCCGGGCCGTTTTAGCTCACAAATGAGTCCGGCCCGTTTCGACGCAC |
| | 13 | ACACCCTCCTAGCACCCGCTGTGCGTCGATCGGGTAATA |
| | 14 | CTGGCCTTCTCAGAACTCTTAGCACGGATTGCGGGTGCT |
| | 15 | TGAGGGAACTTATTACCCGTCCAAATGCGTAGAGTCCCG |
| | 16 | CCAGCCTGGTCGGGACTCTTAGGAGAGCGTAGAGTTCTG |
| 2 | 17 | TGGGTACGCTGTGCAATGTAAAGGCGTGCCGTGATTCGGGTAAGGCTGGAGACTGGATATGGCGAGTTA |
| | 18 | CTTGAACTAATTAGGCAACGGTTTTGCAAGCGCGGCACGCCTTTACATTGCACAGCGTACCCACTCTAT |
| | | ACTGCCCTCCCCCAAGGATTTTTCTCTTAACGCCACTCATGAGCCAGTCTGTATCAAT |
| | 19 | AAGGTGCTAGGCGATTTTTCAGAAGACGCTGTTAGCGAATTCTTAGGTGCCGCGTGCTGCGCCTGCTTG |
| | | CAAAACCGTTGCCTAATTAGTTCAAGCGATGATATCCCACTACCACGATCAACGAGCA |
| | 20 | GTTGGCTCGGGCAAATTGATACAGACTGGCTCATGAGTGGCGTTACCGTGTGGGTAACCGTGTAATCCG |
| | 21 | GAGAAAAATCCTTGGGGGAGGGCAGTATAGAGGGTTGTCACGGGCACGTGCGCTAAAGATCGGTCTGCC |
| | | TCCCGTGGAACCTAGCAACGGTTAAGACGATACACCAGTGGAAATCAAATACTCAGGG |
| | 22 | CTTAACCGTTGCTAGGTTCCACGGGAGGCAGAGACGGGGGGGGCACCGCCCACGATATCCGGGACAGCG |
| | | TCTTCTGAAAAATCGCCTAGCACCTTGCGTGTCCTCTGAAAGCCCACAGCAGCTGGAA |
| | 23 | CCCGGATATCGTGGGCGGTGCCCGCCCCGTCGGACCGACTTCTACCAGTACTACCTAAGATGTCACACC |
| | 24 | TCGATTCTGGTGGCTTCCAGCTGCTGTGGGCTTTCAGAGGACACGCAGGTTCTTGCTAGGACGGCAACG |
| | 25 | CCGATCTTTAGCGCACGTGCCCGTGACAACCATAACTCGCCATATAGGAAAAGCGCGTAGAAGTCGGTC |
| | 26 | GGTTACCCACACGTCCCTGAGTATTTGATTTCCACTGGTGTATCGGCCACCAGAATCGACAGCGCGGTC |
| | 27 | GGCGCAGCACGCGGCACCTAAGAATTCGCTAGGGTGTGACATCTTACTGACGGCATCCTTACCCGAATC |
| | 28 | CTAGCAAGAACCTGTGCTCGTTGATCGTGGTAGTGGGATATCATCATGCCCGAGCCAACCTATGGTGTG |
| | 29 | TGCCGTCAGGGCGCCGCGCTCACACCATATGATCATGCA |
| | 30 | GGTAGTACTAAAGCCAGCTTCGTTGCCGTGGCGCGGCGC |
| | 31 | CAGTCTCCAGTGCATGATCGCGGATTACAACGTCCATCT |
| | 32 | CGCTTTTCCGAGATGGACGCGACCGCGCTCAGCTGGCTT |
| 3 | 33 | TCCCGTACTTCTTTTTTAGTCGAGCAGTGTTGTGTGAGATTGCCGCCACCCAAACTAAATAGGTAATCA |
| | 34 | AGTAACGAAAATTTAGATTATAGTATGGTAACAACACTGCTCGACTAAAAAAGAAGTACGGGACGAGTG |
| | | CTTGAGGCATCCTTCTAATTTTAGTCCCGACGCCGCAGGAGAGACCGAAGGACTAGGG |
| | 35 | CCGGTCAGGCGCATGGCACTGTAGGCATTGTCGTTAGCGATATTCCTACGCACGAGTCAATCACTTACC |
| | | ATACTATAATCTAAATTTTCGTTACTTGCTCACTTCACTACCGCCGGCGGTATGCCAG |
| | 36 | CCTCCGCCTGTCAACCCTAGTCCTTCGGTCTCTCCTGCGGCGTCGTCCATCAACCACCCTTTGGCACTG |
| | 37 | GACTAAAATTAGAAGGATGCCTCAAGCACTCTCGCCTGAGCAGCCTCGCCCGGGGGCATACAGTAGTCC |
| | | GCACGCTTTCCATCCCGGTCTTCGTCGAGTTCACCCCATGAAGAGTAGGGGCCTTTCT |
| | 38 | GACGAAGACCGGGATGGAAAGCGTGCGGACTGGGCTTTCGCTAGGCCCCACCCCCACGGGCTTCACAAT |
| | | GCCTACAGTGCCATGCGCCTGACCGGCCGGAGTGGTGCTTTAATCAAATGCTTGTGTG |
| | 39 | AAGCCCGTGGGGGTGGGGCCTAGCGAAAGCCCCCAACCGAGGCGGCGCAGGTCGATCGAGTCTGCGTGG |
| | 40 | CTAACTGTTCGTATCACACAAGCATTTGATTAAAGCACCACTCCGCGCTGTCCCTCATCACGCGTCCAC |
| | 41 | CTGTATGCCCCCGGGCGAGGCTGCTCAGGCGGTGATTACCTATTTAGGTGCCTTTCCCGCCTCGGTTGG |
| | 42 | GGGTGGTTGATGGCAGAAAGGCCCCTACTCTTCATGGGGTGAACTCTACGAACAGTTAGCCTCAAATCC |
| | 43 | TGATTGACTCGTGCGTAGGAATATCGCTAACCCCACGCAGACTCGCGCCACACACCCGGCAATCTCACA |
| | 44 | GATGAGGGACAGCGCTGGCATACCGCCGGCGGTAGTGAAGTGAGCGTGACAGGCGGAGGCAAAAGTTGC |
| | 45 | GTGTGTGGCGAAGTTACCAGGCAACTTTTGGCCCGCCAG |
| | 46 | TCGACCTGCCCCTGTGGGGCGTGGACGCGCTGGTAACTT |
| | 47 | GTTTGGGTGGCTGGCGGGCCCAGTGCCAACGAGACCAGG |
| | 48 | AAAGGCACCCCCTGGTCTCCGGATTTGAGTCCCCACAGG |

### Example 3: Formation of Other DNA Polyhedra

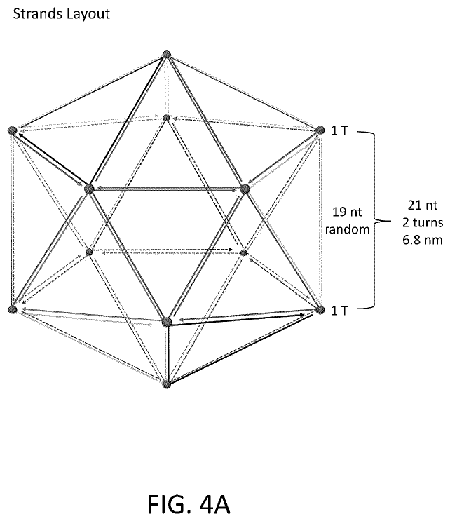

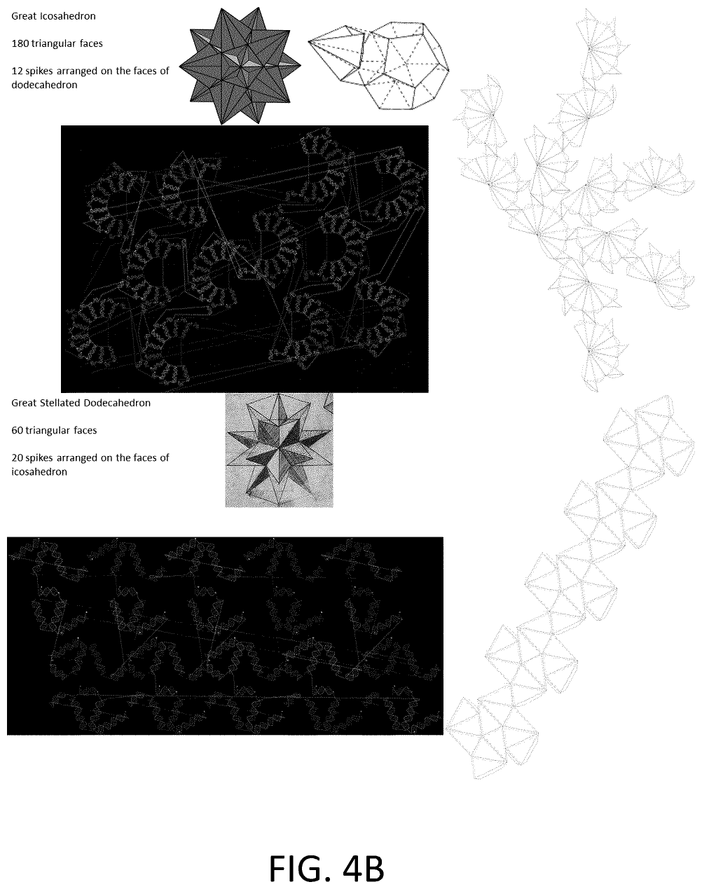

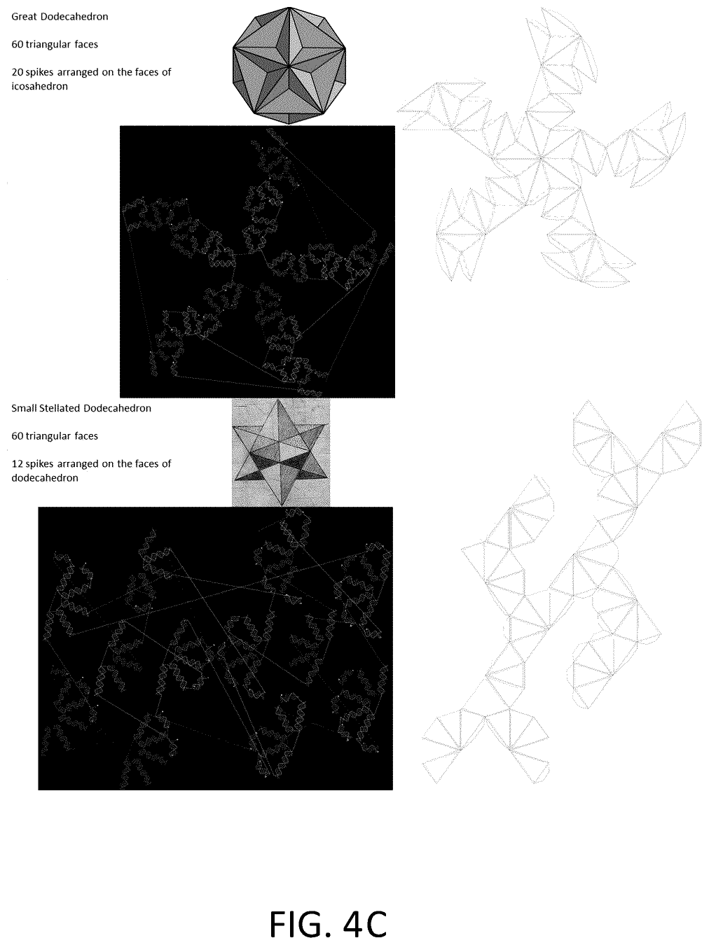

Referring to FIGS. 4A to 4C, these polyhedral shapes, namely icosahedron and Kepler-Poinsot polyhedra, are designed by the same facet-based method with one single-stranded DNA on each face of the structure.

For edge length a of the polyhedron, the volume can be calculated by the formula below:

$$V = \frac{5}{12} ( 3 + \sqrt{5} ) a 3$$

In particular, Table 2 below shows the exemplary sequences of the single-stranded DNAs forming the faces of the icosahedron from 5′ to 3′ end.

TABLE 2

| SEQ | | | | | |
|---|---|---|---|---|---|
| ID | | | | | |
| NO: | | Spacer | | Spacer | |
| 49 | TCATACGGACTAAGGAGGC | T | GATCCTACTATCGGTATAA | T | TGATTAGGAGCGCCCTGGT |
| 50 | CCCCTCGTAGAGAGGAGTG | T | ACCCTTTCGAAAGACGACC | T | TGTATGGGGCGAGGTCAAG |
| 51 | CCCTTGTAGGGGGTCACTT | T | CACTCCTCTCTACGAGGGG | T | CCCGAGCAGTTTTTTGTCT |
| 52 | TCTCAGTGATGGGCTCTCG | T | GTCACCAGCCCTCATCTCC | T | AGACAAAAAACTGCTCGGG |
| 53 | CGAGAGCCCATCACTGAGA | T | GCTGGTGCTTTGGCGTCTG | T | AGGCGGGGGTGACCGTCGT |
| 54 | TGAAGCTATTGGTTGATTG | T | AGGTTTGGCAACCGTGGGA | T | ACGACGGTCACCCCCGCCT |
| 55 | AGAAATTGCGCAGCAGACC | T | CAATCAACCAATAGCTTCA | T | CGTTCATAACCTCCACTTT |
| 56 | CCTGATCGTTTTGGCCCAC | T | GGGCGGCTGGATCCCTTCA | T | AAAGTGGAGGTTATGAACG |
| 57 | GTGGGCCAAAACGATCAGG | T | CAGACGCCAAAGCACCAGC | T | CTTGACCTCGCCCCATACA |
| 58 | CGGATCTTGAGACGCCGGG | T | TGAAGGGATCCAGCCGCCC | T | CTCCAGCGTTCGCCGGCGT |
| 59 | GGTCGTCTTTCGAAAGGGT | T | GCCTCCTTAGTCCGTATGA | T | ACGCCGGCGAACGCTGGAG |
| 60 | ACTCGCCACGCCGAATCTC | T | GCTTCCCGAGCGCACCGTT | T | ACCAGGGCGCTCCTAATCA |
| 61 | GAGATTCGGCGTGGCGAGT | T | GCTTATGTAGGCGAGGCGC | T | GCGGTACCCCTAACGAGTC |
| 62 | CACTCCCTGTTAGACCAAG | T | CTCTGCACGGATACCAGTA | T | GACTCGTTAGGGGTACCGC |
| 63 | TCCCACGGTTGCCAAACCT | T | CTTGGTCTAACAGGGAGTG | T | CTTGTCTACCCTGTGCACG |
| 64 | AACAATGCAGATACAGCCC | T | GGAGATGAGGGCTGGTGAC | T | CGTGCACAGGGTAGACAAG |
| 65 | GGGCTGTATCTGCATTGTT | T | GCGCCTCGCCTACATAAGC | T | TTGCTGGCGCCCGGACGAG |
| 66 | TTATACCGATAGTAGGATC | T | AAGTGACCCCCTACAAGGG | T | CTCGTCCGGGCGCCAGCAA |
| 67 | TACTGGTATCCGTGCAGAG | T | GGTCTGCTGCGCAATTTCT | T | TGCTAAGAGTCTGGACTTC |
| 68 | CCCGGCGTCTCAAGATCCG | T | AACGGTGCGCTCGGGAAGC | T | GAAGTCCAGACTCTTAGCA |

With the above engineered single-stranded DNA components, by using the above-mentioned method, DNA icosahedra and its derivatives, Kepler-Poinsot polyhedra, are reproducibly formed.

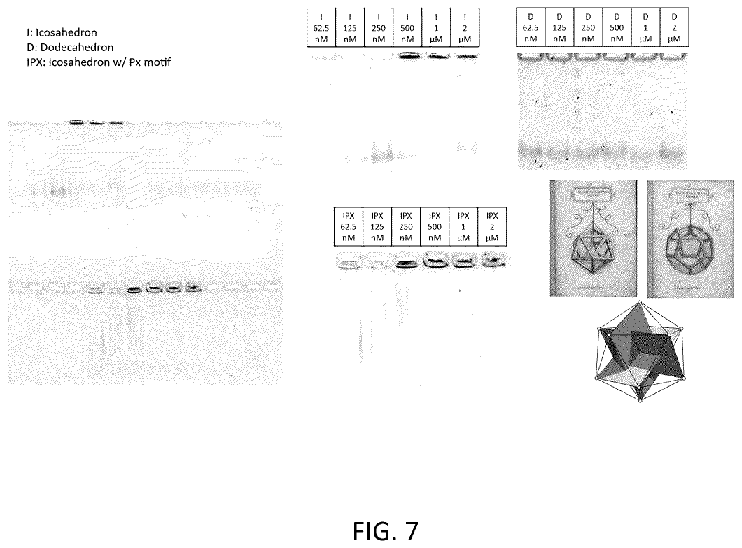

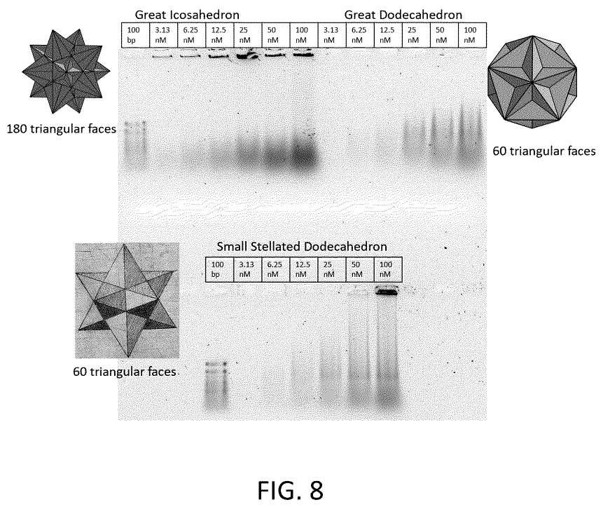

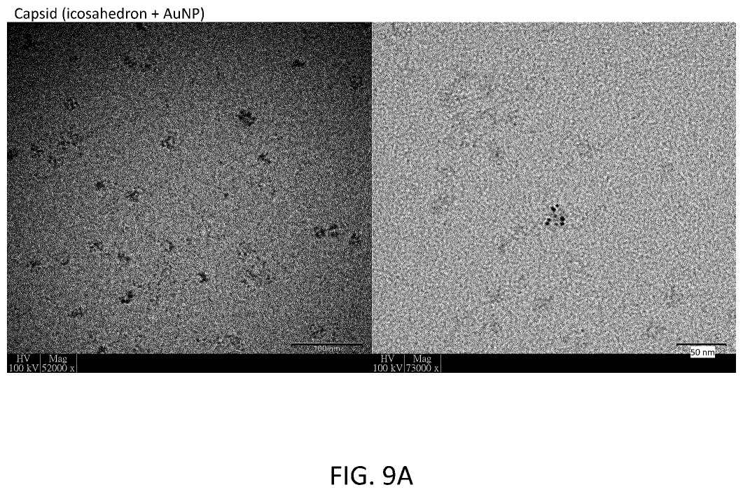

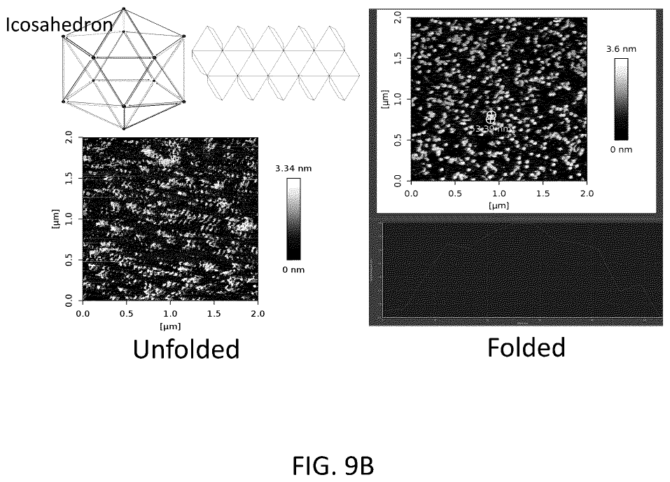

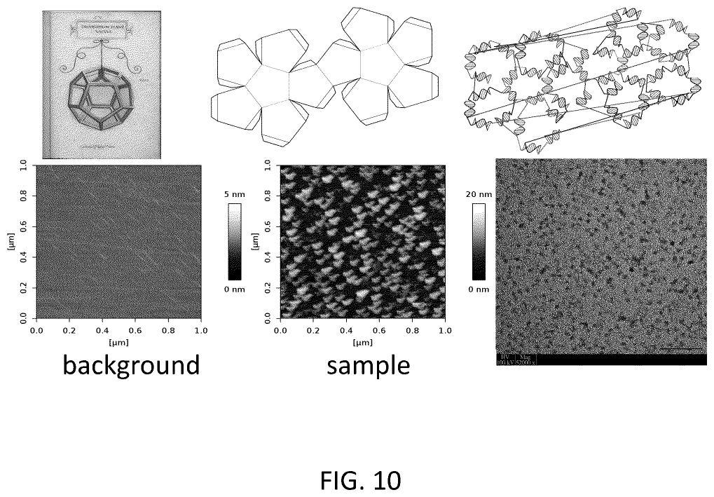

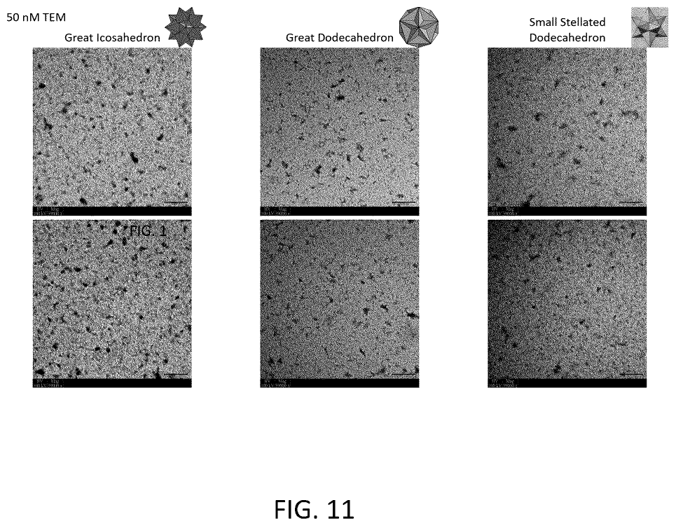

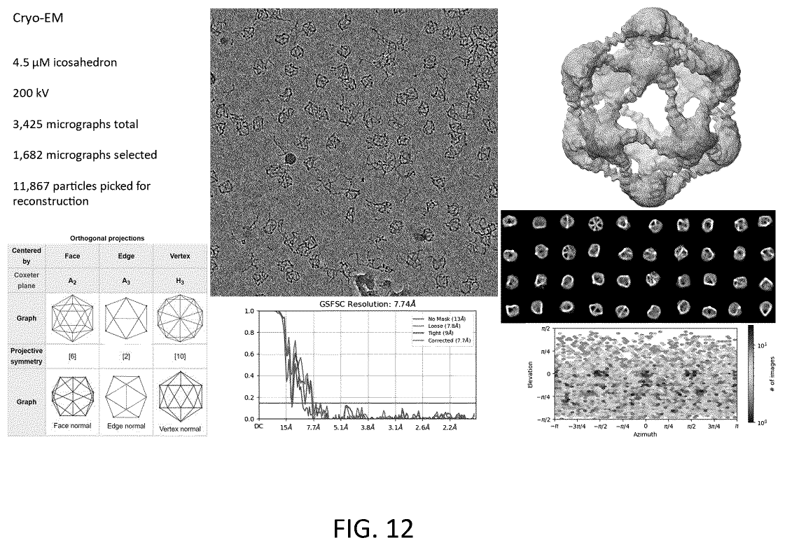

Using the same approach, other complex polyhedra can similarly be assembled using appropriately designed single-stranded DNA components, as supported in FIGS. 7 and 8.

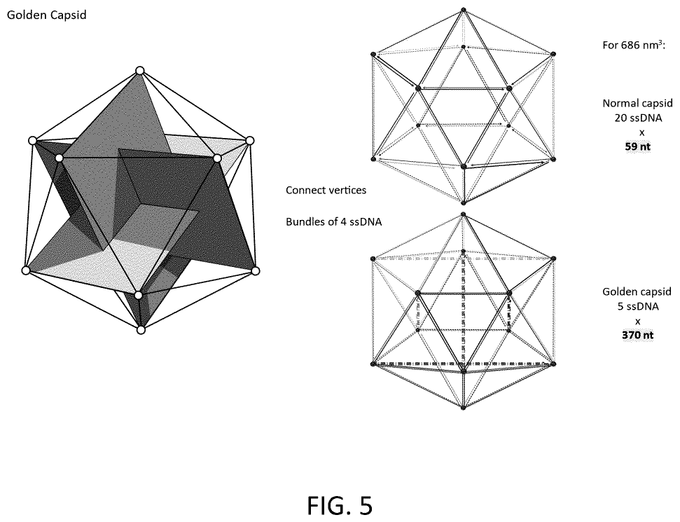

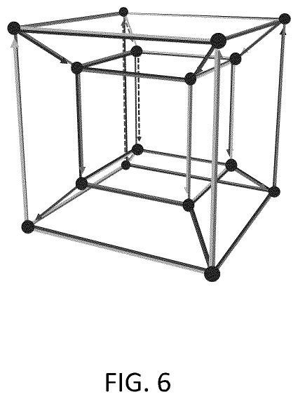

The DNA polyhedra of the present invention can further incorporate paranemic crossover (Px) motif, in which case also helps maintaining the golden ratio of the polyhedral for optimum structural rigidity. See FIGS. 5-7.

### Example 4: Biomedical Applications

The three-dimensional cavity of the DNA polyhedra can be tailored via the strand length of the single-stranded DNA s to encapsulate various payloads, including small molecule drugs, enzymes or imaging agents.

Kepler-Poinsot polyhedra feature spiky protrusions, which enhance cellular membrane penetration and thereby improving drug delivery efficacy.

The method of the present invention offers a simplified alternative to DNA origami. This method reduces assembly complexity while maintaining structural integrity, making the design suitable for therapeutic delivery of agents such as doxorubicin.

In terms of diagnostics, the DNA polyhedra could be extended with aptamer sequences for structural switching detection by forming quadruplexes to generate colorimetric or fluorescent signal. The target could be any protein or small molecule biomarkers. The DNA polyhedra are also adaptable for improving voltammetry electrochemical sensing using electrode with rough surface.

The sizes of the DNA polyhedra are tunable to fit specific surface topographies to enhance signal generation. For example, methylene blue tags may be added for electrochemical detection.

The DNA polyhedra are also applicable in positron emission tomography (PET) scan and magnetic resonance imaging (MRI) scan. Similar to the principle of targeted drug delivery for cancer therapeutics, the DNA polyhedra of the present invention has the capability to load the small and radioactive tracer for PET and MRI scan such as 2-deoxy-2-18F-fluoro-β-D-glucose (18F-FDG).

Several embodiments of the present disclosure and features of details are briefly described above. The embodiments described in the present disclosure may be easily used as a basis for designing or modifying other processes and structures for realizing the same or similar objectives and/or obtaining the same or similar advantages introduced in the embodiments of the present disclosure. Such equivalent construction does not depart from the spirit and scope of the present disclosure, and various variations, replacements, and modifications can be made without departing from the spirit and scope of the present disclosure.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of +10%, +5%, +1%, or +0.5% of the given value or range. The range may be indicated herein as from one endpoint to another endpoint or between two endpoints. Unless otherwise specified, all the ranges disclosed in the present disclosure include endpoints. The term “substantially coplanar” may refer to two surfaces within a few micrometers (μm) positioned along the same plane, for example, within 10 μm, within 5 μm, within 1 μm, or within 0.5 μm located along the same plane. When reference is made to “substantially” the same numerical value or characteristic, the term may refer to a value within ±10%, ±5%, ±1%, or ±0.5% of the average of the values.

1. A method for constructing a polyhedral DNA nanostructure, comprising:
a) providing a plurality of single-stranded DNA (ssDNA) oligonucleotides, each corresponding to a distinct face of a polygonal or polyhedral structure;
b) configuring each ssDNA oligonucleotide with one or more edge hybridization domains at terminal regions of the strand;
c) designing said edge hybridization domains to be complementary only to domains on ssDNA oligonucleotides corresponding to adjacent faces;
d) mixing the ssDNA oligonucleotides in a hybridization buffer under conditions that allow selective hybridization between adjacent edge domains;
thereby forming a closed, staple-free and scaffold-free polyhedral DNA nanostructure.

2. The method of claim 1, wherein the shape of the polyhedral DNA nanostructure is selected from icosahedron, dodecahedron, tesseract, a Kepler-Poinsot polyhedron, or a Platonic polyhedron.

3. The method of claim 1, wherein the polyhedral DNA nanostructure has a melting temperature of at least 80° C.

4. The method of claim 1, wherein the polyhedral DNA nanostructure further comprises inner paranemic crossover (Px) motifs.

5. A DNA-based polyhedral nanostructure comprising:
a first single-stranded DNA (ssDNA) oligonucleotide configured to define a first face of a polygonal or polyhedral structure;
a second ssDNA oligonucleotide configured to define a second face adjacent to the first face;
wherein the first ssDNA oligonucleotide comprises an edge hybridization domain located at a first edge of the first face;
wherein the second ssDNA oligonucleotide comprises a complementary edge hybridization domain located at a corresponding edge of the second face;
wherein the edge hybridization domain of the first ssDNA hybridizes with the complementary edge domain of the second ssDNA to join the first and second faces;
and wherein each ssDNA oligonucleotide in the structure is uniquely configured to hybridize only with adjacent ssDNA oligonucleotides corresponding to the directly adjoining faces of the polyhedron.

6. The DNA-based polyhedral nanostructure of claim 5, wherein the DNA-based polyhedral nanostructure is free from staples.

7. A DNA-based polyhedral nanostructure for delivering a therapeutic agent to a target site in a subject, comprising:
the DNA-based polyhedral nanostructure of claim 5;
an internal cavity defined by the DNA-based polyhedral nanostructure; and
a therapeutic agent;
wherein the therapeutic agent is encapsulated within the internal cavity; and
wherein the polyhedral framework further comprises a targeting moiety for the therapeutic agent selected from an antibody, an aptamer, a carbohydrate or a peptide ligand.

8. The DNA-based polyhedral nanostructure of claim 7, wherein the therapeutic agent is selected from a chemotherapeutic agent, a nucleic acid-based therapeutic, a protein or peptide drug, a vaccine antigen or an immunomodulatory molecule.

9. A DNA-based biomolecular diagnostic platform, comprising:
the DNA-based polyhedral nanostructure of claim 5;
a detection moiety bound to a portion of the DNA-based polyhedral nanostructure; and
a detection system configured to measure a signal produced by the DNA-based polyhedral nanostructure upon binding to one or more target biomolecules.

10. The platform of claim 9, wherein the detection moiety is selected from a fluorophore, a quencher, a biotin tag or a molecular beacon.

11. A method for detecting a target biomolecule in a sample using the platform of claim 9, comprising:
introducing the DNA-based polyhedral nanostructure into the sample; and
detecting a signal change indicating the presence of a target biomolecule according to the detection moiety.


SEQUENCE LISTING (68 sequences)

SEQ ID NO: 1 - RNA, 69 nt, synthetic construct
  source 1..69: mol_type = other RNA
tagggcgcccgttaaccatggcgccgagcgatgggcaaaaaagcctgttccctcatgaatgggaacggt

SEQ ID NO: 2 - DNA, 127 bp, synthetic construct
  source 1..127: mol_type = other DNA
gttggaacggtcgtggcacgcgtcgccgtcattcgctcggcgccatggttaacgggcgccctatgggaggcggtgccgtcacatgccctgctccttggttaggcccgaatttcggcatgtcacgcgg

SEQ ID NO: 3 - DNA, 127 bp, synthetic construct
  source 1..127: mol_type = other DNA
tgccgaggaatgagcgcaggtgtcgaacacttgctgcgctctaatttccggagttagcgccacttgacggcgacgcgtgccacgaccgttccaacttttgtgagctaaaacggcccgggcttgcatc

SEQ ID NO: 4 - DNA, 69 bp, synthetic construct
  source 1..69: mol_type = other DNA
acgggccggactctccgcgtgacatgccgaaattcgggcctaacctgggccggtgcccttcgcatttgg

SEQ ID NO: 5 - DNA, 127 bp, synthetic construct
  source 1..127: mol_type = other DNA
aggagcagggcatgtgacggcaccgcctccctagggaaagcctgtgtacgttacagtcatctgtgaccacacgattctgtgctggtcccgggccgtggcacataccgacaagccataccgcgccacg

SEQ ID NO: 6 - DNA, 127 bp, synthetic construct
  source 1..127: mol_type = other DNA
cggcccgggaccagcacagaatcgtgtggtctggatactaatcggtcaggtgggcccgagtgatagtgttcgacacctgcgctcattcctcggcatcttcaaaattgggcttgaagctagctgtgtg

SEQ ID NO: 7 - DNA, 69 bp, synthetic construct
  source 1..69: mol_type = other DNA
tcactcgggcccacctgaccgattagtatcctaggagagccggtgtgaaggccagtcacggcgccgtaa

SEQ ID NO: 8 - DNA, 69 bp, synthetic construct
  source 1..69: mol_type = other DNA
acgtcggcgtggctcacacagctagcttcaagcccaattttgaagtacgatagtagagctatccgtgct

SEQ ID NO: 9 - DNA, 69 bp, synthetic construct
  source 1..69: mol_type = other DNA
cagatgactgtaacgtacacaggctttcccttaccgttcccattctccaggctggtcaccggctctcct

SEQ ID NO: 10 - DNA, 69 bp, synthetic construct
  source 1..69: mol_type = other DNA
agggcaccggccctcgtggcgcggtatggcttgtcggtatgtgcctgccacgccgacgttcgctctcct

SEQ ID NO: 11 - DNA, 69 bp, synthetic construct
  source 1..69: mol_type = other DNA
gtggcgctaactccggaaattagagcgcagctttacggcgccgtgtggagggtgttggcttttttgccc

SEQ ID NO: 12 - DNA, 69 bp, synthetic construct
  source 1..69: mol_type = other DNA
gctctactatcgttgatgcaagcccgggccgttttagctcacaaatgagtccggcccgtttcgacgcac

SEQ ID NO: 13 - DNA, 39 bp, synthetic construct
  source 1..39: mol_type = other DNA
acaccctcctagcacccgctgtgcgtcgatcgggtaata

SEQ ID NO: 14 - DNA, 39 bp, synthetic construct
  source 1..39: mol_type = other DNA
ctggccttctcagaactcttagcacggattgcgggtgct

SEQ ID NO: 15 - DNA, 39 bp, synthetic construct
  source 1..39: mol_type = other DNA
tgagggaacttattacccgtccaaatgcgtagagtcccg

SEQ ID NO: 16 - DNA, 39 bp, synthetic construct
  source 1..39: mol_type = other DNA
ccagcctggtcgggactcttaggagagcgtagagttctg

SEQ ID NO: 17 - DNA, 69 bp, synthetic construct
  source 1..69: mol_type = other DNA
tgggtacgctgtgcaatgtaaaggcgtgccgtgattcgggtaaggctggagactggatatggcgagtta

SEQ ID NO: 18 - DNA, 127 bp, synthetic construct
  source 1..127: mol_type = other DNA
cttgaactaattaggcaacggttttgcaagcgcggcacgcctttacattgcacagcgtacccactctatactgccctcccccaaggatttttctcttaacgccactcatgagccagtctgtatcaat

SEQ ID NO: 19 - DNA, 127 bp, synthetic construct
  source 1..127: mol_type = other DNA
aaggtgctaggcgatttttcagaagacgctgttagcgaattcttaggtgccgcgtgctgcgcctgcttgcaaaaccgttgcctaattagttcaagcgatgatatcccactaccacgatcaacgagca

SEQ ID NO: 20 - DNA, 69 bp, synthetic construct
  source 1..69: mol_type = other DNA
gttggctcgggcaaattgatacagactggctcatgagtggcgttaccgtgtgggtaaccgtgtaatccg

SEQ ID NO: 21 - DNA, 127 bp, synthetic construct
  source 1..127: mol_type = other DNA
gagaaaaatccttgggggagggcagtatagagggttgtcacgggcacgtgcgctaaagatcggtctgcctcccgtggaacctagcaacggttaagacgatacaccagtggaaatcaaatactcaggg

SEQ ID NO: 22 - DNA, 127 bp, synthetic construct
  source 1..127: mol_type = other DNA
cttaaccgttgctaggttccacgggaggcagagacggggcgggcaccgcccacgatatccgggacagcgtcttctgaaaaatcgcctagcaccttgcgtgtcctctgaaagcccacagcagctggaa

SEQ ID NO: 23 - DNA, 69 bp, synthetic construct
  source 1..69: mol_type = other DNA
cccggatatcgtgggcggtgcccgccccgtcggaccgacttctaccagtactacctaagatgtcacacc

SEQ ID NO: 24 - DNA, 69 bp, synthetic construct
  source 1..69: mol_type = other DNA
tcgattctggtggcttccagctgctgtgggctttcagaggacacgcaggttcttgctaggacggcaacg

SEQ ID NO: 25 - DNA, 69 bp, synthetic construct
  source 1..69: mol_type = other DNA
ccgatctttagcgcacgtgcccgtgacaaccataactcgccatataggaaaagcgcgtagaagtcggtc

SEQ ID NO: 26 - DNA, 69 bp, synthetic construct
  source 1..69: mol_type = other DNA
ggttacccacacgtccctgagtatttgatttccactggtgtatcggccaccagaatcgacagcgcggtc

SEQ ID NO: 27 - DNA, 69 bp, synthetic construct
  source 1..69: mol_type = other DNA
ggcgcagcacgcggcacctaagaattcgctagggtgtgacatcttactgacggcatccttacccgaatc

SEQ ID NO: 28 - DNA, 69 bp, synthetic construct
  source 1..69: mol_type = other DNA
ctagcaagaacctgtgctcgttgatcgtggtagtgggatatcatcatgcccgagccaacctatggtgtg

SEQ ID NO: 29 - DNA, 39 bp, synthetic construct
  source 1..39: mol_type = other DNA
tgccgtcagggcgccgcgctcacaccatatgatcatgca

SEQ ID NO: 30 - DNA, 39 bp, synthetic construct
  source 1..39: mol_type = other DNA
ggtagtactaaagccagcttcgttgccgtggcgcggcgc

SEQ ID NO: 31 - DNA, 39 bp, synthetic construct
  source 1..39: mol_type = other DNA
cagtctccagtgcatgatcgcggattacaacgtccatct

SEQ ID NO: 32 - DNA, 39 bp, synthetic construct
  source 1..39: mol_type = other DNA
cgcttttccgagatggacgcgaccgcgctcagctggctt

SEQ ID NO: 33 - DNA, 69 bp, synthetic construct
  source 1..69: mol_type = other DNA
tcccgtacttcttttttagtcgagcagtgttgtgtgagattgccgccacccaaactaaataggtaatca

SEQ ID NO: 34 - DNA, 127 bp, synthetic construct
  source 1..127: mol_type = other DNA
agtaacgaaaatttagattatagtatggtaacaacactgctcgactaaaaaagaagtacgggacgagtgcttgaggcatccttctaattttagtcccgacgccgcaggagagaccgaaggactaggg

SEQ ID NO: 35 - DNA, 127 bp, synthetic construct
  source 1..127: mol_type = other DNA
ccggtcaggcgcatggcactgtaggcattgtcgttagcgatattcctacgcacgagtcaatcacttaccatactataatctaaattttcgttacttgctcacttcactaccgccggcggtatgccag

SEQ ID NO: 36 - DNA, 69 bp, synthetic construct
  source 1..69: mol_type = other DNA
cctccgcctgtcaaccctagtccttcggtctctcctgcggcgtcgtccatcaaccaccctttggcactg

SEQ ID NO: 37 - DNA, 127 bp, synthetic construct
  source 1..127: mol_type = other DNA
gactaaaattagaaggatgcctcaagcactctcgcctgagcagcctcgcccgggggcatacagtagtccgcacgctttccatcccggtcttcgtcgagttcaccccatgaagagtaggggcctttct

SEQ ID NO: 38 - DNA, 127 bp, synthetic construct
  source 1..127: mol_type = other DNA
gacgaagaccgggatggaaagcgtgcggactgggctttcgctaggccccacccccacgggcttcacaatgcctacagtgccatgcgcctgaccggccggagtggtgctttaatcaaatgcttgtgtg

SEQ ID NO: 39 - DNA, 69 bp, synthetic construct
  source 1..69: mol_type = other DNA
aagcccgtgggggtggggcctagcgaaagcccccaaccgaggcggcgcaggtcgatcgagtctgcgtgg

SEQ ID NO: 40 - DNA, 69 bp, synthetic construct
  source 1..69: mol_type = other DNA
ctaactgttcgtatcacacaagcatttgattaaagcaccactccgcgctgtccctcatcacgcgtccac

SEQ ID NO: 41 - DNA, 69 bp, synthetic construct
  source 1..69: mol_type = other DNA
ctgtatgcccccgggcgaggctgctcaggcggtgattacctatttaggtgcctttcccgcctcggttgg

SEQ ID NO: 42 - DNA, 69 bp, synthetic construct
  source 1..69: mol_type = other DNA
gggtggttgatggcagaaaggcccctactcttcatggggtgaactctacgaacagttagcctcaaatcc

SEQ ID NO: 43 - DNA, 69 bp, synthetic construct
  source 1..69: mol_type = other DNA
tgattgactcgtgcgtaggaatatcgctaaccccacgcagactcgcgccacacacccggcaatctcaca

SEQ ID NO: 44 - DNA, 69 bp, synthetic construct
  source 1..69: mol_type = other DNA
gatgagggacagcgctggcataccgccggcggtagtgaagtgagcgtgacaggcggaggcaaaagttgc

SEQ ID NO: 45 - DNA, 39 bp, synthetic construct
  source 1..39: mol_type = other DNA
gtgtgtggcgaagttaccaggcaacttttggcccgccag

SEQ ID NO: 46 - DNA, 39 bp, synthetic construct
  source 1..39: mol_type = other DNA
tcgacctgcccctgtggggcgtggacgcgctggtaactt

SEQ ID NO: 47 - DNA, 39 bp, synthetic construct
  source 1..39: mol_type = other DNA
gtttgggtggctggcgggcccagtgccaacgagaccagg

SEQ ID NO: 48 - DNA, 39 bp, synthetic construct
  source 1..39: mol_type = other DNA
aaaggcaccccctggtctccggatttgagtccccacagg

SEQ ID NO: 49 - DNA, 59 bp, synthetic construct
  source 1..59: mol_type = other DNA
tcatacggactaaggaggctgatcctactatcggtataattgattaggagcgccctggt

SEQ ID NO: 50 - DNA, 59 bp, synthetic construct
  source 1..59: mol_type = other DNA
cccctcgtagagaggagtgtaccctttcgaaagacgaccttgtatggggcgaggtcaag

SEQ ID NO: 51 - DNA, 59 bp, synthetic construct
  source 1..59: mol_type = other DNA
cccttgtagggggtcactttcactcctctctacgaggggtcccgagcagttttttgtct

SEQ ID NO: 52 - DNA, 59 bp, synthetic construct
  source 1..59: mol_type = other DNA
tctcagtgatgggctctcgtgtcaccagccctcatctcctagacaaaaaactgctcggg

SEQ ID NO: 53 - DNA, 59 bp, synthetic construct
  source 1..59: mol_type = other DNA
cgagagcccatcactgagatgctggtgctttggcgtctgtaggcgggggtgaccgtcgt

SEQ ID NO: 54 - DNA, 59 bp, synthetic construct
  source 1..59: mol_type = other DNA
tgaagctattggttgattgtaggtttggcaaccgtgggatacgacggtcacccccgcct

SEQ ID NO: 55 - DNA, 59 bp, synthetic construct
  source 1..59: mol_type = other DNA
agaaattgcgcagcagacctcaatcaaccaatagcttcatcgttcataacctccacttt

SEQ ID NO: 56 - DNA, 59 bp, synthetic construct
  source 1..59: mol_type = other DNA
cctgatcgttttggcccactgggcggctggatcccttcataaagtggaggttatgaacg

SEQ ID NO: 57 - DNA, 59 bp, synthetic construct
  source 1..59: mol_type = other DNA
gtgggccaaaacgatcaggtcagacgccaaagcaccagctcttgacctcgccccataca

SEQ ID NO: 58 - DNA, 59 bp, synthetic construct
  source 1..59: mol_type = other DNA
cggatcttgagacgccgggttgaagggatccagccgccctctccagcgttcgccggcgt

SEQ ID NO: 59 - DNA, 59 bp, synthetic construct
  source 1..59: mol_type = other DNA
ggtcgtctttcgaaagggttgcctccttagtccgtatgatacgccggcgaacgctggag

SEQ ID NO: 60 - DNA, 59 bp, synthetic construct
  source 1..59: mol_type = other DNA
actcgccacgccgaatctctgcttcccgagcgcaccgtttaccagggcgctcctaatca

SEQ ID NO: 61 - DNA, 59 bp, synthetic construct
  source 1..59: mol_type = other DNA
gagattcggcgtggcgagttgcttatgtaggcgaggcgctgcggtacccctaacgagtc

SEQ ID NO: 62 - DNA, 59 bp, synthetic construct
  source 1..59: mol_type = other DNA
cactccctgttagaccaagtctctgcacggataccagtatgactcgttaggggtaccgc

SEQ ID NO: 63 - DNA, 59 bp, synthetic construct
  source 1..59: mol_type = other DNA
tcccacggttgccaaaccttcttggtctaacagggagtgtcttgtctaccctgtgcacg

SEQ ID NO: 64 - DNA, 59 bp, synthetic construct
  source 1..59: mol_type = other DNA
aacaatgcagatacagccctggagatgagggctggtgactcgtgcacagggtagacaag

SEQ ID NO: 65 - DNA, 59 bp, synthetic construct
  source 1..59: mol_type = other DNA
gggctgtatctgcattgtttgcgcctcgcctacataagctttgctggcgcccggacgag

SEQ ID NO: 66 - DNA, 59 bp, synthetic construct
  source 1..59: mol_type = other DNA
ttataccgatagtaggatctaagtgaccccctacaagggtctcgtccgggcgccagcaa

SEQ ID NO: 67 - DNA, 59 bp, synthetic construct
  source 1..59: mol_type = other DNA
tactggtatccgtgcagagtggtctgctgcgcaatttctttgctaagagtctggacttc

SEQ ID NO: 68 - DNA, 59 bp, synthetic construct
  source 1..59: mol_type = other DNA
cccggcgtctcaagatccgtaacggtgcgctcgggaagctgaagtccagactcttagca